(19) United States
(12) Patent Application Publication
(10) Pub. No.: US 2025/0090298 A1
(43) Pub. Date: Mar. 20, 2025

(54) CARDIOVASCULAR IMPLANT DEVICES WITH FLOW CONDITIONERS TO MINIMIZE DISRUPTION TO AND ENHANCE CARDIOVASCULAR HEMODYNAMICS

(71) Applicant: EDWARDS LIFESCIENCES CORPORATION, Irvine, CA (US)

(72) Inventors: Brandon Sina Dehdashtian, Irvine, CA (US); Mazen Saadi Albaghdadi, Irvine, CA (US)

(21) Appl. No.: 18/969,649

(22) Filed: Dec. 5, 2024

Related U.S. Application Data
(63) Continuation of application No. PCT/US2023/024703, filed on Jun. 7, 2023.
(60) Provisional application No. 63/349,824, filed on Jun. 7, 2022.

(51) Int. Cl. A61F 2/02

(52) U.S. Cl. CPC A61F 2/02; A61F 2210/0009; A61F 2210/0014; A61F 2210/0057; A61F 2230/0006; A61F 2250/0004; A61F 2250/0039; A61F 2250/0069

(57) ABSTRACT

A cardiovascular implant device includes an expandable annular frame and a flow conditioner. The expandable annular frame is formed of a plurality of struts and is configured to conform to an interior shape of a blood vessel or a chamber of a heart when expanded inside the blood vessel or the chamber of the heart. The flow conditioner is connected to the plurality of struts of the expandable annular frame. The flow conditioner is positioned to modify a hemodynamic characteristic of a flow of blood through or out of the expandable annular frame.

### CROSS-REFERENCE TO RELATED APPLICATIONS

This application is a continuation of International Application No. PCT/US2023/024703, filed Jun. 7, 2023, which claims the benefit of U.S. Provisional Application No. 63/349,824, filed Jun. 7, 2022, the disclosures of which are hereby incorporated by reference in their entireties.

### BACKGROUND

The present disclosure relates to cardiovascular implant devices, and more specifically to cardiovascular implant devices for minimizing disruption to and enhancing cardiovascular hemodynamics.

Various medical devices can be implanted in the cardiovascular system at sites where blood will flow through or around the implanted device. For example, prosthetic or artificial valve devices can be deployed to replace native valves that are diseased or malfunctioning. Edge-to-edge valve repair devices are also deployed to native valve sites to treat tricuspid regurgitation (TR) or mitral regurgitation (MR). Additionally, stents can be deployed into the cardiovascular system to hold open a stenosed vessel. These implantable devices can be placed in natural flow paths within the cardiovascular system.

### SUMMARY

In one example, a cardiovascular implant device includes an expandable annular frame and a flow conditioner. The expandable annular frame is formed of a plurality of struts and is configured to conform to an interior shape of a blood vessel or a chamber of a heart when expanded inside the blood vessel or the chamber of the heart. The flow conditioner is connected to the plurality of struts of the expandable annular frame. The flow conditioner is positioned to modify a hemodynamic characteristic of a flow of blood through or out of the expandable annular frame.

In another example, a prosthetic valve device includes an annular frame formed of a plurality of struts, a valvular body mounted within the annular frame, and a flow conditioner. The valvular body includes a plurality of leaflets that regulate a flow of blood through the annular frame. The flow conditioner is connected to the plurality of struts of the annular frame. The flow conditioner is positioned to modify a hemodynamic characteristic of the flow of blood through or out of the annular frame.

In another example, a prosthetic valve system includes a present device having a frame with a bi-directionally flared profile that is formed of a first plurality of struts, a prosthetic valve device configured to sit within the present device, a first flow conditioner, and a second flow conditioner. The prosthetic valve device includes an annular frame formed of a second plurality of struts and a valvular body mounted within the annular frame. The valvular body includes a plurality of leaflets that regulate a flow of blood through the annular frame. The first flow conditioner is connected to the first plurality of struts of the present device. The first flow conditioner is positioned to modify a first hemodynamic characteristic of the flow of blood through or out of the present device. The second flow conditioner is connected to the second plurality of struts of the prosthetic valve device. The second flow conditioner is positioned to modify a second hemodynamic characteristic of the flow of blood through or out of the prosthetic valve device.

In another example, a cardiovascular implant device includes a body and a flow conditioner connected to the body. The body is configured to attach to one or more leaflets of a natural heart valve. The body includes a central spacer and clasps extending radially outward from the central spacer. Each of the clasps includes a first arm and a second arm for gripping the one or more leaflets. The flow conditioner is positioned to modify a hemodynamic characteristic of a flow of blood around the cardiovascular implant device.

### BRIEF DESCRIPTION OF THE DRAWINGS

FIG. 1 is a schematic diagram of a heart and vasculature.

FIG. 2A is a first schematic diagram illustrating modeled hemodynamic flow patterns in a heart.

FIG. 2B is a second schematic diagram illustrating modeled hemodynamic flow patterns in a heart.

FIG. 3A is a perspective view of a first example of a cardiovascular implant device including fin-type flow conditioners.

FIG. 3B is a top view of the first example of the cardiovascular implant device including the fin-type flow conditioners.

FIG. 3C is a bottom view of the first example of the cardiovascular implant device including the fin-type flow conditioners.

FIG. 4 is a sectional view of a heart illustrating an example positioning at a non-valve site of the first example of the cardiovascular implant device including the fin-type flow conditioners.

FIG. 5A is an enlarged partial perspective view illustrating a fin-type flow conditioner interacting with low blood flow through the first example of the cardiovascular implant device.

FIG. 5B is an enlarged partial perspective view illustrating the fin-type flow conditioner interacting with high blood flow through the first example of the cardiovascular implant device.

FIG. 6A is a schematic diagram illustrating connection of control components to an actively controlled flow conditioner.

FIG. 6B is a schematic diagram illustrating electromechanical actuation of the actively controlled flow conditioner.

FIG. 7 is a sectional view of a heart illustrating an example positioning of the control components for the actively controlled flow conditioner of FIGS. 6A-6B.

FIG. 8 is a schematic diagram illustrating an example control system for the actively controlled flow conditioners of FIGS. 6A-6B.

FIGS. 9A-9E are enlarged partial perspective views of a frame of the first example of the cardiovascular implant device illustrating several variations of fin-type flow conditioners.

FIG. 10A is a perspective view of a second example of a cardiovascular implant device including plate-type flow conditioners.

FIG. 10B is a top view of the second example of the cardiovascular implant device including the plate-type flow conditioners.

FIG. 10C is a bottom view of the second example of the cardiovascular implant device including the plate-type flow conditioners.

FIGS. 11A-11E are perspective views of the second example of the cardiovascular implant device illustrating several variations of plate-type flow conditioners.

FIG. 12 is a perspective view of a third example of a cardiovascular implant device including fin-type flow conditioners.

FIG. 13 is a sectional view of a heart illustrating an example positioning at a valve site of the third example of the cardiovascular implant device including the fin-type flow conditioners.

FIG. 14 is a perspective view of a fourth example of a cardiovascular implant device including a plate-type flow conditioner.

FIG. 15A is a perspective view of a fifth example of a cardiovascular implant device including fin-type flow conditioners.

FIG. 15B is a top view of the fifth example of the cardiovascular implant device including the fin-type flow conditioners.

FIG. 15C is a bottom view of the fifth example of the cardiovascular implant device including the fin-type flow conditioners.

FIG. 16A is a sectional view of a heart illustrating an example positioning at a valve site of the fifth example of the cardiovascular implant device including the fin-type flow conditioners.

FIG. 16B is a sectional view of a heart illustrating an example positioning at a non-valve site of the fifth example of the cardiovascular implant device including the fin-type flow conditioners.

FIG. 17A is a perspective view of a sixth example of a cardiovascular implant device including plate-type flow conditioners.

FIG. 17B is a top view of the sixth example of the cardiovascular implant device including the plate-type flow conditioners.

FIG. 17C is a bottom view of the sixth example of the cardiovascular implant device including the plate-type flow conditioners.

FIG. 18 is a sectional view of a heart illustrating an example positioning of a seventh example of a cardiovascular implant device including fin-type flow conditioners.

FIG. 19 is a sectional view of a heart illustrating an example positioning of an eighth example of a cardiovascular implant device including a plate-type flow conditioner.

FIG. 20 is a sectional view of a heart illustrating an example positioning of a ninth example of a cardiovascular implant device including fin-type flow conditioners.

FIG. 21 is a flowchart showing a method for selecting a cardiovascular implant device including a flow conditioner for implantation in the heart.

### DETAILED DESCRIPTION

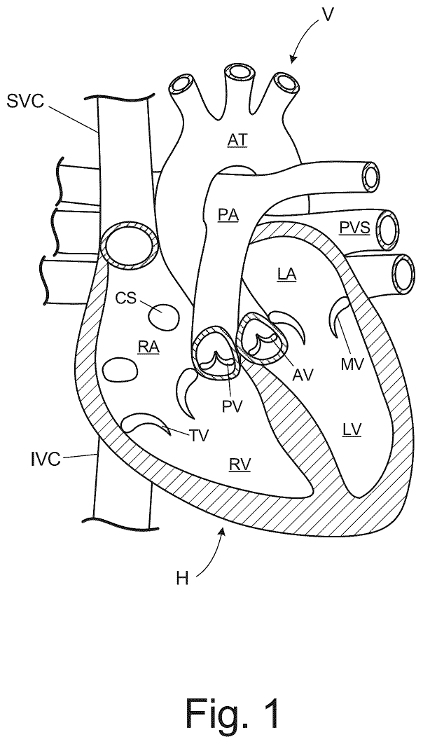

FIG. 1 is a schematic diagram of heart H and vasculature V. FIG. 1 shows heart H, vasculature V, right atrium RA, right ventricle RV, left atrium LA, left ventricle LV, superior vena cava SVC, inferior vena cava IVC, tricuspid valve TV, pulmonary valve PV, pulmonary artery PA, pulmonary veins PVS, mitral valve MV, aortic valve AV, aorta AT, and coronary sinus CS.

Heart H is a human heart that receives blood from and delivers blood to vasculature V. Heart H includes four chambers: right atrium RA, right ventricle RV, left atrium LA, and left ventricle LV.

The right side of heart H, including right atrium RA and right ventricle RV, receives deoxygenated blood from vasculature V and pumps the blood to the lungs. Blood flows into right atrium RA from superior vena cava SVC, inferior vena cava IVC, and coronary sinus CS.

A majority of the blood flows into right atrium RA from superior vena cava SVC and inferior vena cava IVC, which are offset from one another. Due to the offset of the major entry blood flows from superior vena cava SVC and inferior vena cava IVC, a natural flow vortex occurs in right atrium RA (a right-sided flow vortex). This allows a substantial portion of blood from right atrium RA to pass through right atrium RA and enter right ventricle RV by direct flow. The right-sided flow vortex in right atrium RA preserves kinetic energy and momentum of the major blood flows entering right atrium RA and allows a substantial portion of blood to naturally pass from right atrium RA to right ventricle RV without any contribution to flow needed from the pumping action of right atrium RA. With contraction, right atrium RA also pumps the residual portion of the entering blood not caught in the direct flow through tricuspid valve TV into right ventricle RV. The blood enters right ventricle RV and then flows through pulmonary valve PV into pulmonary artery PA. With preservation of direct inflow from right atrium RA, blood entering right ventricle RV also forms a natural flow vortex (a right-ventricular flow vortex) in right ventricle RV, which naturally re-directs blood entering right ventricle RV to pulmonary artery PA by direct flow without requiring right ventricle RV to perform substantial work of pumping blood. Residual blood that is not transported to pulmonary artery PA via pulmonary valve PV by direct flow is pumped by the contraction of right ventricle RV. The blood flows from pulmonary artery PA into smaller arteries that deliver the deoxygenated blood to the lungs via the pulmonary circulatory system. The lungs can then oxygenate the blood.

The left side of heart H, including left atrium LA and left ventricle LV, receives the oxygenated blood from the lungs and provides blood flow to the body. Blood flows into left atrium LA from pulmonary veins PVS. The offset of the right and left pulmonary veins PVS also leads to the formation of a natural flow vortex in left atrium LA (left-sided flow vortex), which helps maintain momentum and minimize work as the blood traverses left atrium LA to mitral valve MV. Direct flow, as described above, and the pumping action of left atrium LA propels the blood through mitral valve MV into left ventricle LV. As the blood enters left ventricle LV, a natural flow vortex (a left-ventricular flow vortex) forms in left ventricle LV, which redirects flow naturally towards the left ventricular outflow of aortic valve AV so that it can be efficiently pumped by left ventricle LV through aortic valve AV into aorta AT. The blood flows from aorta AT into arteries that deliver the oxygenated blood to the body via the systemic circulatory system.

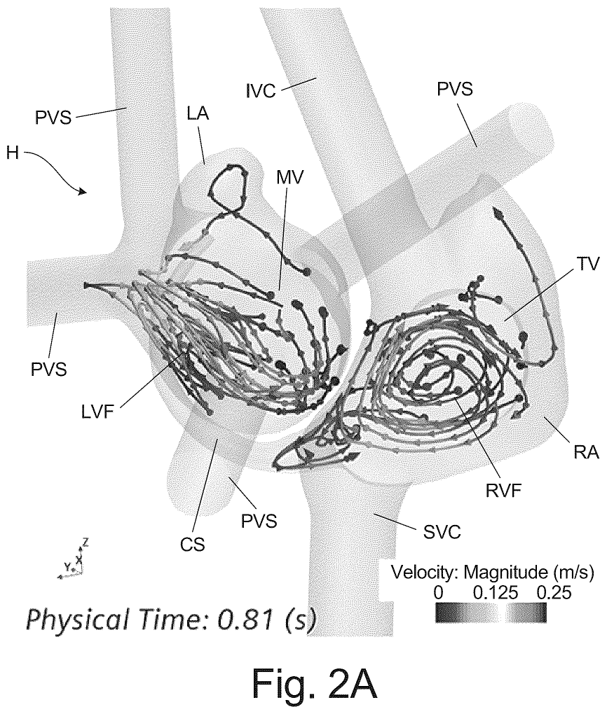

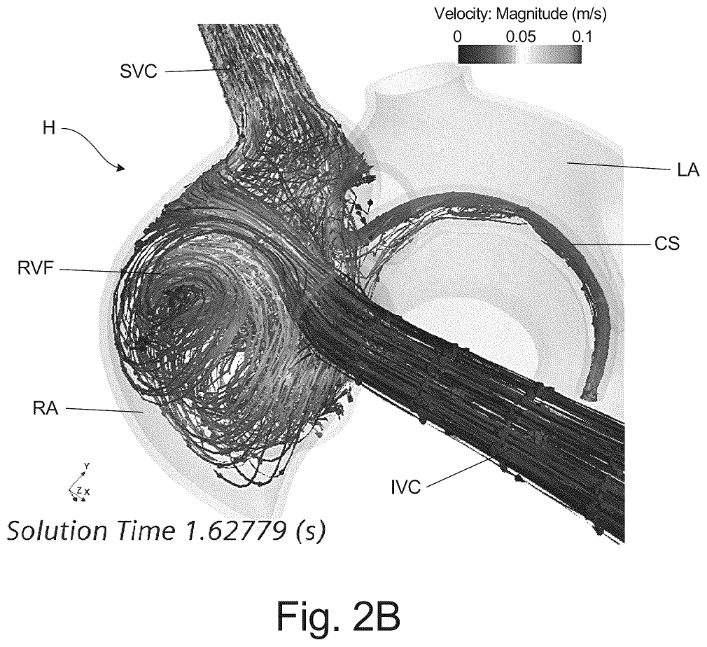

FIG. 2A is a first schematic diagram illustrating modeled hemodynamic flow patterns in heart H. FIG. 2B is a second schematic diagram illustrating modeled hemodynamic flow patterns in heart H. FIGS. 2A-2B show heart H, right atrium RA, left atrium LA, superior vena cava SVC, inferior vena cava IVC, and coronary sinus CS. FIG. 2A shows tricuspid valve TV, pulmonary veins PVS, and mitral valve MV.

FIGS. 2A-2B show modeled velocity stream lines representing hemodynamic flow patterns in heart H. FIG. 2A shows heart H oriented with right atrium RA on a right side of the figure and left atrium LA on a left side of the figure. FIG. 2A is an inferior view of heart H. FIG. 2B shows heart H oriented with right atrium RA on a left side of the figure and left atrium LA on a right side of the figure. FIG. 2B is a superior view of heart H.

Natural flow patterns of blood flow exist in heart H and help move blood through heart H and into the vasculature connected to heart H in a way that maximizes preservation of blood flow momentum and kinetic energy. The natural flow pattern for blood moving through arteries and veins is typically helical in nature (helical flow patterns). The natural flow pattern for blood moving through the chambers of heart H is typically vortical in nature (vortical flow patterns).

FIG. 2A shows modeled hemodynamic flow patterns that exist in right atrium RA and left atrium LA of heart H. FIG. 2B shows modeled hemodynamic flow patterns that exist in right atrium RA, superior vena cava SVC, inferior vena cava IVC, and coronary sinus CS. FIGS. 2A-2B represent natural flow patterns that are formed in heart H, including right atrium RA and left atrium LA, based on the offset inflows of blood into the chambers of heart H in addition to the anatomical structure of heart H. When looking at heart H from the right side (the right sagittal view), a clockwise right-sided flow vortex is formed in right atrium RA and a counter-clockwise left-sided flow vortex is formed in left atrium LA. The right-sided flow vortex in right atrium RA is the natural flow pattern of blood flow in right atrium RA. The left-sided flow vortex in left atrium LA is the natural flow pattern of blood flow in left atrium LA. The modeled hemodynamic flow patterns shown in FIGS. 2A-2B represent intra-cardiac flow patterns for a structurally normal heart.

Blood flows enter the right atrium RA from superior vena cava SVC, inferior vena cava IVC, and coronary sinus CS. The superior vena cava opening and the inferior vena cava opening in right atrium RA are offset so that the blood flowing into right atrium RA from superior vena cava SVC and inferior vena cava IVC do not collide with each other. Due to its orientation and physical proximity, coronary sinus CS flow is entrained into inferior vena cava IVC flow. The blood flowing through superior vena cava SVC and inferior vena cava IVC has a helical flow pattern. A majority of the blood in right atrium RA enters right atrium RA through inferior vena cava IVC, and the blood flowing from inferior vena cava IVC into right atrium RA is pointed towards the top of right atrium RA. The helical flow pattern of the blood flowing into right atrium RA from inferior vena cava IVC helps to form a clockwise right-sided flow vortex in right atrium RA (when looking at the heart from the right side). The flow of blood entering right atrium RA from superior vena cava SVC will flow along the inter-atrial septum and towards tricuspid valve TV. The helical flow pattern of the blood flowing from superior vena cava SVC into right atrium RA helps the flow of blood naturally join with the clockwise right-sided flow vortex formed in right atrium RA by the flow of blood from inferior vena cava IVC, which is joined by coronary sinus CS flow. A small amount of blood flows into right atrium RA from coronary sinus CS. The flow flowing through coronary sinus CS will have a helical flow pattern. The helical flow pattern of the blood exiting coronary sinus CS will naturally join with inferior vena cava IVC flow and the right-sided flow vortex in right atrium RA. The right-sided flow vortex in right atrium RA is shown with velocity stream lines labeled RVF in FIGS. 2A-2B.

The right-sided flow vortex formed in right atrium RA helps the blood flow through right atrium RA, through tricuspid valve TV, into the right ventricle, through the pulmonary valve, and into the pulmonary artery. The right heart is an inefficient pump and can act more like a conduit. The right-sided flow vortex formed in the right heart helps to preserve kinetic energy and the momentum of blood flow as it moves from superior vena cava SVC and inferior vena cava IVC (the Vena Cavac) through the right heart and into the pulmonary artery, even with minimal to no pumping being provided by the right heart. This is especially important for maintaining right heart output, which must match left heart output, during periods of high output and heart rates during exercise. The right-sided flow vortex formed in right atrium RA helps to move the blood from right atrium RA through tricuspid valve TV and into the right ventricle with minimal loss of momentum and kinetic energy. The blood shoots from right atrium RA through the right ventricle, out the right ventricular outflow tract, through the pulmonary valve, and into the pulmonary artery. Approximately 50% of the blood will flow into the pulmonary artery without any pumping required by the right heart because of the right-sided flow vortices of right atrium RA and right ventricle RV and anatomical constraints of the right heart. Right heart contraction enhances the flow of residual blood through the right heart.

Blood flows into left atrium LA from pulmonary veins PVS. There are four pulmonary veins PVS that flow into left atrium LA. The blood flowing through pulmonary veins PVS has a helical flow pattern. The offset of helical flow of the blood flowing from pulmonary veins PVS into left atrium LA helps to form a counter-clockwise left-sided flow vortex (when looking at the heart from the right side) in left atrium LA. The left-sided flow vortex in left atrium LA directs flow towards mitral valve MV. The left-sided flow vortex in left atrium LA is shown with velocity stream lines labeled LVF in FIG. 2A.

Although not illustrated in FIG. 2A-2B, blood flowing through aorta AT from left ventricle LV can also have characteristic helical and vortical flow patterns. Similar characteristic helical and/or vortical flow patterns may also be present in the other vessels (e.g., pulmonary artery PA) or chambers (e.g., left ventricle LV and right ventricle RV) of heart H.

It is hypothesized that if the intra-cardiac blood flow patterns in heart H (including right-sided flow vortex in right atrium RA and left-sided flow vortex in left atrium LA) are disrupted, blood flow from superior vena cava SVC and inferior vena cava IVC (the Vena Cavac), through right atrium RA, through the right ventricle, and into the pulmonary artery, and blood flow from the pulmonary veins, through the left atrium LA, through the left ventricle, and into the aorta become less efficient and place increased mechanical workloads on the respective ventricles. This is especially important in already failing hearts, where the ability to increase the workload of the heart muscle is impaired. Disruptions in the intra-cardiac blood flow patterns in heart H (including right-sided flow vortex in right atrium RA and left-sided flow vortex in left atrium LA) can happen for a variety of reasons. For example, the anatomy of heart H can change as patients age. This can affect the offset between the opening of superior vena cava SVC and the opening of inferior vena cava IVC. The blood flow entering right atrium RA from superior vena cava SVC and the blood flow entering right atrium RA from inferior vena cava IVC can collide as the anatomy of heart H changes, which disrupts the natural formation of the right-sided flow vortex in right atrium RA. In another example, right atrium RA can be enlarged in patients with heart failure with or without atrial fibrillation. The enlargement of right atrium RA can also disrupt the right-sided flow vortex formed in right atrium RA. Similarly, left atrium LA can be enlarged in patients with heart failure with or without atrial fibrillation. The enlargement of left atrium LA can disrupt the left-sided flow vortex formed in left atrium LA. Additionally, patients with a patent foramen ovale (a natural inter-atrial septal shunt) or a secundum atrial septal defect due to failure of the patent foramen ovale to fully close may not have the expected intra-cardiac blood flow patterns (including right-sided flow vortex in right atrium RA and left-sided flow vortex in left atrium LA), including the expected flow vortexes created during atrial filling. Closure of a secundum atrial septal defect with altered right atrial non-single vortex flow patterns has been shown to revert to a dominant single vortical flow pattern after the atrial septal defect is occluded.

In another example, the introduction of implant devices, such as valves or stents, to the cardiovascular anatomy can also disrupt the natural flow patterns in the vessels or chambers of heart H in which or near where the device is implanted, such as the right-sided flow vortex in right atrium RA and the left-sided flow vortex in left atrium LA. This can be due to mismatches (however slight) between the artificial flow path through the implanted device and the natural flow path that has been replaced. For example, aortic vortical and/or helical flow can be disrupted after transcatheter aortic valve replacement (TAVR). Pre-TAVR, helical flow in the aorta can move the blood downstream through the aorta. Post-TAVR, vortical flows may have a more pronounced effect, which can result in less energy toward forward (downstream) movement in the aortic flow. Moreover, blood flowing through an implanted device will tend to hug to the walls of the device. If the device structure ends abruptly at a downstream end of the device, the flow of blood will immediately separate from the edge and become turbulent flow, which in turn causes flow reversal and thereby decreased hemodynamic efficiency.

When the right-sided flow vortex in right atrium RA changes, momentum and energy of the blood flow are lost and the right heart needs to pump harder to move the blood from right atrium RA into the right ventricle and the pulmonary artery. This is due to the right-sided flow vortex contributing less to the movement of blood through the right heart. Similarly, when the left-sided flow vortex in left atrium LA changes, the left heart needs to pump harder to move the blood from left atrium LA into the left ventricle and the aorta. This is due to the left-sided flow vortex contributing less to the movement of blood through the left heart. Further, as the intra-cardiac flow patterns in heart H (including right-sided flow vortex in right atrium RA and left-sided flow vortex in left atrium LA) change due to age or disease, areas of turbulence can be created in the flow patterns of heart H and there can be a loss of fluid dynamics leading to inefficiencies that could lead to diminished flow. This can increase the susceptibility of the right heart and/or the left heart to fail (the inability to pump enough blood to meet the body's oxygen demands), as heart H has to do more work to move the same amount of blood through heart H. More work is needed to recreate the lost momentum naturally preserved by the intra-cardiac flow patterns in heart H (including right-sided flow vortex in right atrium RA and left-sided flow vortex in left atrium LA), putting additional strain on heart H. Hemodynamic efficiency of valves in heart H also play a role in the work required by heart H. Small changes or inefficiencies in hemodynamics caused by diseased or malfunctioning valves, or the presence of artificial valve devices, when multiplied by tens of thousands of beats can result in significant unnecessary energy consumption by heart H.

Changes in intra-cardiac flow patterns change intra-cardiac energetics. Heart H is uniquely designed to maximize efficiency by preserving the kinetic energy and momentum of blood flow, thus minimizing the work needed to propagate the blood flow into the chambers, between the chambers, and out of the chambers. Anything that disrupts the intra-cardiac flow patterns in heart H (including right-sided flow vortex in right atrium RA and left-sided flow vortex in left atrium LA) can reduce the efficiency of the energetics of heart H due to a loss of potential energy, which makes it more difficult for heart H to do its job of propagating blood into, between, and out of the chambers. Anything that disrupts the intra-cardiac flow patterns through heart H (including right-sided flow vortex in right atrium RA and left-sided flow vortex in left atrium LA) can increase the amount of work heart H has to do, prolong transit times through heart H, and makes it more difficult for heart H to eject blood. This is especially problematic for people experiencing heart failure, as the heart failure can be exacerbated due to disruptions in the intra-cardiac flow patterns through heart H (including right-sided flow vortex in right atrium RA and left-sided flow vortex in left atrium LA).

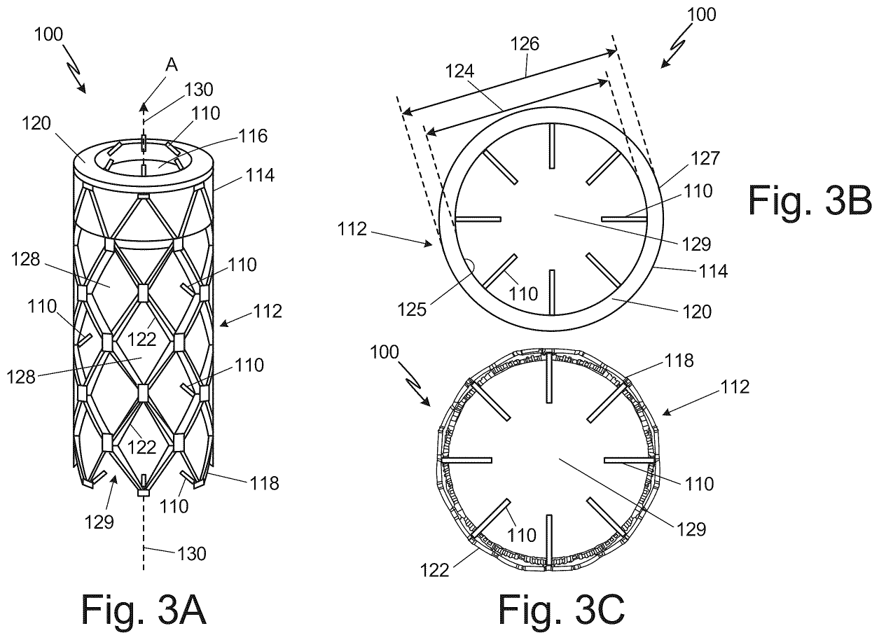

### DEVICE 100 (FIGS. 3A-9E)

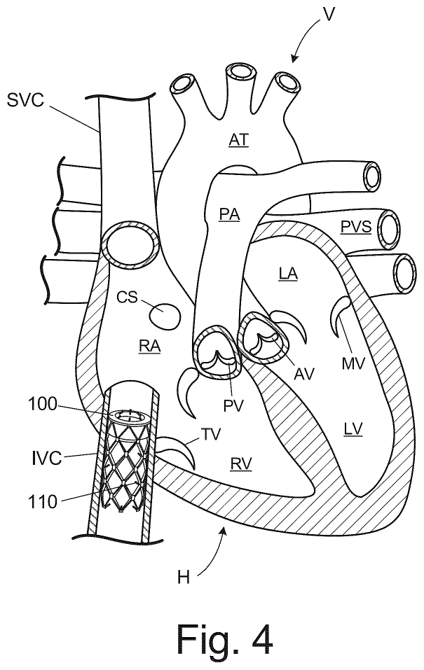

FIGS. 3A-4 will be described together. FIG. 3A is a perspective view of cardiovascular implant device 100 including fin-type flow conditioners 110. FIG. 3B is a top view of cardiovascular implant device 100 including fin-type flow conditioners 110. FIG. 3C is a bottom view of cardiovascular implant device 100 including fin-type flow conditioners 110. FIG. 4 is a sectional view of heart H illustrating an example positioning at a non-valve site of cardiovascular implant device 100 including fin-type flow conditioners 110.

As illustrated in FIGS. 3A-3C, cardiovascular implant device 100 includes fin-type flow conditioners 110, frame 112, cover 114, valve seat 116, inflow end 118, and outflow end 120. Frame 112 includes struts 122, inner diameter 124, inner surface 125, outer diameter 126, and outer surface 127, and defines openings 128, central flow path 129, and flow axis 130. FIG. 4 also shows device 100, heart H, vasculature V, right atrium RA, right ventricle RV, left atrium LA, left ventricle LV, superior vena cava SVC, inferior vena cava IVC, tricuspid valve TV, pulmonary valve PV, pulmonary artery PA, pulmonary veins PVS, mitral valve MV, aortic valve AV, aorta AT, and coronary sinus CS.

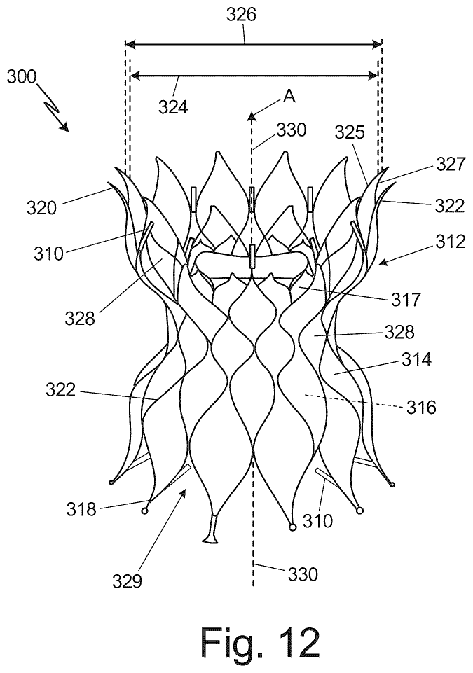

Cardiovascular implant device 100 is an implantable device for use in a cardiovascular system. Cardiovascular implant device 100 is configured to be implanted in blood vessels or chambers of heart H. In the illustrated example, cardiovascular implant device 100 is a “prestent” or docking station for supporting a valve device, such as a prosthetic valve device. Cardiovascular implant device 100 can be delivered into the cardiovascular system via a catheter (i.e., transcatheter delivery) or can be surgically placed using transcatheter or surgical procedures known in the art. As shown in FIG. 4, device 100 is located in inferior vena cava IVC near its opening into right atrium RA. That is, device 100 is located at a site in heart H where there is not naturally a valve (a “non-valve” site). In other examples, device 100 can be located in superior vena cava SVC. In yet other examples, device 100 can be located in any vessel or chamber of heart H at a non-valve site or at a site where there is a natural valve (e.g., aortic valve AV, mitral valve MV, pulmonary valve PV, etc.). Examples of a cardiovascular implant device in a valve site are described below with reference to FIGS. 12-14.

Frame 112 forms a main body of device 100. Frame 112 can be expandable. Frame 112 can have a wide variety of different shapes and sizes. As shown in FIGS. 3A-3C, frame 112 is an annular or cylindrical mesh or lattice. Frame 112 has inner diameter 124 and outer diameter 126. Each of inner diameter 124 and outer diameter 126 can vary along a length of frame 112. Inner diameter 124 is the diameter of radially inner surface 125 of frame 112. Outer diameter 126 is the diameter of radially outer surface 127 of frame 112. Frame 112 can have any suitable length. For example, frame 112 may be approximately as long as a valve that is configured to sit within frame 112 (e.g., within valve seat 116). In other examples, frame 112 can be longer or shorter than a valve that is configured to sit within frame 112. Frame 112 can press against or into tissue walls at the implant site or contour (or extend) around anatomical structures of the cardiovascular system to set and maintain the position of device 100.

Frame 112 can be formed in a variety of ways, e.g., connecting individual wires together to form a mesh or lattice, braiding, cutting from a sheet and then rolling or otherwise forming into the shape of frame 112, molding, cutting from a cylindrical tube (e.g., cutting from a nitinol tube), other ways, or a combination of these. Frame 112 can be made from a highly flexible metal, metal alloy, or polymer. Examples of metals and metal alloys that can be used include, but are not limited to, nitinol and other shape-memory alloys, elgiloy, and stainless steel, but other metals and highly resilient or compliant non-metal materials can be used to make frame 112. All or a portion of frame 112 can be monolithically formed of any of these materials. These materials can allow frame 112 to be compressed to a small size, and then-when the compression force is released-frame 112 can self-expand back to its pre-compressed shape. Frame 112 can expand back to its pre-compressed shape due to the material properties frame 112 is made of and/or frame 112 can be expanded by inflation or expansion of a device positioned inside frame 112. For example, frame 112 can be compressed such that frame 112 can fit into a delivery catheter. Frame 112 can also be made of other materials and can be expandable and collapsible in different ways, e.g., mechanically-expandable, balloon-expandable, self-expandable, or a combination of these.

Frame 112 extends between inflow end 118 and outflow end 120 of cardiovascular implant device 100. Inflow end 118 can be an end of device 100 that is relatively upstream of outflow end 120 with respect to a flow of blood along flow axis 130, as represented by arrow A in FIG. 3A, when device 100 is implanted in a blood vessel or chamber of heart H. Accordingly, outflow end 120 is an end of device 100 that is relatively downstream of inflow end 118 with respect to a flow of blood along flow axis 130, as represented by arrow A in FIG. 3A, when device 100 is implanted in a blood vessel or chamber of heart H. In the example shown in FIG. 4, outflow end 120 is positioned near where inferior vena cava IVC opens into right atrium RA and inflow end 118 is positioned upstream within inferior vena cava IVC. Although inflow end 118 is defined as being relatively upstream of outflow end 120, it should be understood that other actual positions of inflow end 118 or outflow end 120 are possible depending on the location where device 100 is implanted.

Frame 112 is formed of a plurality of struts 122. Struts 122 make up the lattice or mesh of frame 112 and define openings (or cells) 128 therein. Struts 122 can be integrally formed. In some examples, all or a portion of struts 122 are monolithically formed from the same material. Openings 128 extend through frame 112 from inner surface 125 to outer surface 127. Each of openings 128 is bounded on one or more sides by ones of struts 122. Openings 128 can have any suitable shape or size, which can in turn be based on an overall shape or size of frame 112. In the example shown in FIG. 3A, openings 128 are diamond shaped and arranged in circumferential rows around frame 112. In other examples, openings 128 can have any other regular or irregular polygonal or non-polygonal shape and pattern. In some examples, ones of openings 128 can have different shapes or sizes throughout frame 112. In some examples, ones of openings 128 can be connected by gaps to adjacent ones of openings 128.

Central flow path 129 is an open channel through a central portion of annular frame 112. Central flow path 129 is defined by inner surface 125 of frame 112. Central flow path 129 extends from inflow end 118 to outflow end 120 such that device 100 is open at each end. Accordingly, blood flowing through and out of device 100 follows central flow path 129. More specifically, flow axis 130 is a longitudinal axis through device 100 along which blood flows as it passes or is directed through device 100 (e.g., in the direction indicated by arrow A in FIG. 3A). In the example illustrated in FIG. 4 where device 100 is implanted in inferior vena cava IVC, a valve seated in device 100 can open when heart H is in a diastolic phase. Blood flows from inferior vena cava IVC and superior vena cava SVC into right atrium RA. The blood that flows from inferior vena cava IVC flows through device 100 along flow axis 130. During the diastolic phase, blood in right atrium RA flows through tricuspid valve TV and into right ventricle RV. During a systolic phase of heart H, a valve seated in device 100 can close. Blood is prevented from flowing (i.e., backflowing) from right atrium RA into inferior vena cava IVC by the closed valve in device 100. A closed valve in device 100 prevents any blood that regurgitates through the through tricuspid valve TV during the systolic phase from being forced into inferior vena cava IVC.

Cover 114 is a covering for one or more portions of frame 112. Cover 114 can be a fabric material, a polymer material, or other material. For example, cover 114 can be a material that promotes tissue ingrowth where device 100 contacts adjacent tissue walls of a vessel or chamber of heart H. Cover 114 can also form a seal to limit or prevent blood flow through portions of frame 112 that are covered by cover 114. Cover 114 can be attached to frame 112 by any suitable attachment means, such as by stitching, gluing, tying, etc. Cover 114 can be shaped and positioned in a variety of ways. In the example shown in FIG. 3A, cover 114 is adjacent to outflow end 120. In some examples, cover 114 is near or adjacent valve seat 116. In other examples, cover 114 can be adjacent to inflow end 118 or at any location or locations between inflow end 118 and outflow end 120. In yet other examples, device 100 does not include cover 114.

Valve seat 116 is a portion of device 100 for holding, supporting, or attaching to a valve device, such as a prosthetic valve device. In some examples, valve seat 116 can be a portion of frame 112. In some examples, valve seat 116 can be monolithically formed with frame 112. In other examples, valve seat 116 can be formed separately from frame 112 and attached. Valve seat 116 can take any form that provides a supporting surface for implanting or deploying a valve within device 100 after device 100 is implanted in the cardiovascular system. In the example shown in FIG. 3A, valve seat 116 is located near outflow end 120. However, it should be understood that in other examples valve seat 116 can be located at any lengthwise position along frame 112. Valve seat 116 (and a valve seated therein, not shown) can span across a portion of central flow path 129. Valve seat 116 allows a valve to be implanted in vasculature or tissue of varying strengths, sizes, and shapes. The outer profile of device 100 (e.g., outer surface 127 of frame 112) can better conform to the cardiovascular anatomy (e.g., vasculature, tissue, heart, etc.) without putting too much pressure on the anatomy, while a valve can be firmly and securely implanted in valve seat 116 to prevent or mitigate the risk of migration or slipping.

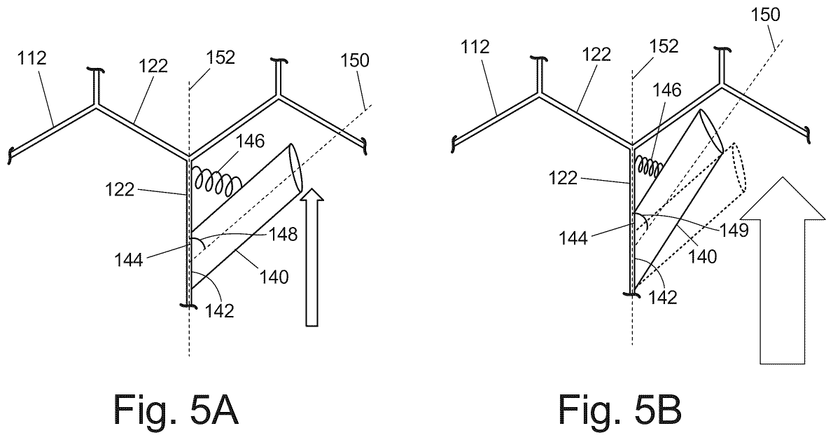

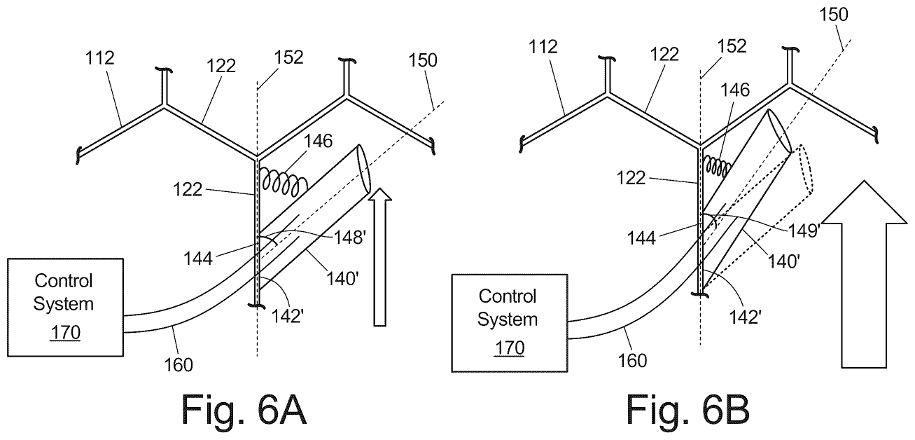

Flow conditioners 110 are fins or fin-type flow conditioners. Each individual one of flow conditioners 110 can also be referred to as a flow conditioner feature. Flow conditioners 110 are elongated projections from frame 112. More specifically, flow conditioners 110 are connected to frame 112 at corresponding ones of struts 122. Flow conditioners 110 are attached by an attachment mechanism (as described in greater detail with reference to FIGS. 5A-5B below) or monolithically formed with a portion of frame 112 (as described in greater detail with reference to FIGS. 6A-6B below).

In general, flow conditioners 110 can take a number of different forms (i.e., shapes, sizes, etc.). In some examples, flow conditioners 110 can be airfoils. Flow conditioners 110 can have a symmetrical or asymmetrical and regular or irregular shape and can have variable geometries. Physical dimensions (e.g., length, width, shape, cross-sectional shape, etc.) of flow conditioners 110 can be configured to prevent flow conditioners 110 from interfering (or contacting) other parts of device 100 or adjacent tissue walls. In other examples, components of device 100 (e.g., cover 114) can be designed to fit around flow conditioners 110 or to permit flow conditioners 110 to pass through. The physical dimension of flow conditioners 110 can further be configured to allow flow conditioners 110 to collapse and expand with expandable frame 112 (e.g., to fit within a delivery catheter). The physical dimensions of flow conditioners 110 can further be configured to prevent flow conditioners 110 from occluding a vessel of chamber of heart H in which device 100 is implanted. That is, a length and/or width of flow conditioners 110 can be relatively short enough so that flow conditioners 110 do not protrude from device 100 and extend fully across a vessel or chamber of heart H and block blood flow. The physical dimensions of flow conditioners 110 can further be configured to modify a hemodynamic characteristic (e.g., helicity, vorticity, velocity, turbulence, flow direction, etc.) of blood flowing through or out of device 100 in a particular manner.

One or more flow conditioners 110 can be positioned in any suitable arrangement with respect to frame 112 of device 100. In some examples, flow conditioners 110 can be arranged about a circumference of inner surface 125 of frame 112. In some examples, flow conditioners 110 can be located adjacent inflow end 118 (as shown in FIG. 3C) and/or outflow end 120 (as shown in FIG. 3B). In some examples, flow conditioners 110 are connected to ones of struts 122 that form a first row of openings 128 that is adjacent to outflow end 120. Device 100 can include any number of flow conditioners 110 in any one or more of the foregoing locations. Locations of flow conditioners 110 can be configured to prevent flow conditioners 110 from interfering (or contacting) other parts of device 100 or adjacent tissue walls. In other examples, components of device 100 (e.g., cover 114) can be designed to fit around flow conditioners 110 or to permit flow conditioners 110 to pass through. The locations of flow conditioners 110 can further be configured to allow flow conditioners 110 to collapse and expand with expandable frame 112 (e.g., to fit within a delivery catheter). The locations of flow conditioners 110 can further be configured to prevent flow conditioners 110 from occluding a vessel of chamber of heart H in which device 100 is implanted. The locations of flow conditioners 110 can further be configured to modify a hemodynamic characteristic (e.g., helicity, vorticity, velocity, turbulence, flow direction, etc.) of blood flowing through or out of device 100 in a particular manner.

Once device 100 is implanted in cardiovascular system (e.g., in inferior vena cava IVC as shown in FIG. 4), circulating blood passes through device 100. As blood flows into, through, and out of device 100 along flow axis 130, flow conditioners 110 interact with the blood flow to modify or affect a hemodynamic characteristic (e.g., helicity, vorticity, velocity, turbulence, flow direction, etc.) of the flow. Flow conditioners 110 can interact with blood flowing through or out of device 100 by adding flow resistance and/or changing the direction of the blood flow to prevent reversal of blood flow. For example, flow conditioners 110 may increase or decrease vorticity or helicity of the flow. In some examples, flow conditioners 110 may cause the flow to be smoother (decrease the turbulence). In other examples, flow conditioners 110 can increase turbulence in the flow. In some examples, flow conditioners 110 can change a flow direction of the flow. In some examples, flow conditioners 110 can align the flow with a natural vortical flow pattern of blood through a vessel or chamber of heart H, such as the left-sided flow vortex in left atrium LA or the right-sided flow vortex in right atrium RA. In other examples, flow conditioners 110 can align the flow with a natural helical flow pattern of blood through a vessel or chamber of heart H, such as helical flow in coronary sinus CS. Flow conditioners 110 that are located circumferentially at inflow end 118 and/or outflow end 120 of device 100 can create helical flow patterns near an adjacent vessel or chamber wall to force blood to flow back towards the center of the vessel or chamber in a same helical direction, thereby producing forward movement of the blood. More generally, flow conditioners 110 adjacent to inflow end 118 can modify a hemodynamic characteristic of blood flowing through frame 112, and flow conditioners 110 adjacent to outflow end 120 can modify a hemodynamic characteristic of blood flowing out of frame 112.

Cardiovascular implant device 100, including flow conditioners 110, can produce hemodynamic effects to minimize disruption to or enhance the natural flow patterns in heart H, such as the left-sided flow vortex in left atrium LA, the right-sided flow vortex in right atrium RA, and/or helical flow in coronary sinus CS. Flow conditioners 110 can modify hemodynamic characteristics of blood flowing through or out of device 100 such that (a) any disruptions to the natural flow patterns that would be caused by an implantable device are minimized; (b) reduced flow due to a pathophysiology or other cause is mitigated; and/or (c) baseline flow is enhanced. As a result, device 100 can maintain kinetic energy of the cardiovascular blood flow, which in turn reduces the cardiac work needed and improves cardiac efficiency. These hemodynamic effects can potentially improve patient outcomes after receiving cardiovascular implant device 100 because device 100 can be more effective and potentially safer. At the same time, flow conditioners 110 can be incorporated relatively easily into device 100 in many configurations, so different variations of device 100 can be optimized for use in many different scenarios (e.g., for many different patient conditions).

FIGS. 5A-5B will be described together. FIG. 5A is an enlarged partial perspective view illustrating fin-type flow conditioner 140 interacting with low blood flow through cardiovascular implant device 100. FIG. 5B is an enlarged partial perspective view illustrating fin-type flow conditioner 140 interacting with high blood flow through cardiovascular implant device 100.

FIGS. 5A-5B show frame 112 (of cardiovascular implant device 100) including struts 122, fin-type flow conditioner 140 including attachment region 142, joint 144, and bias member 146. FIGS. 5A-5B also show attachment angle 148 (FIG. 5A), deflection angle 149 (FIG. 5B), fin longitudinal axis 150, and strut longitudinal axis 152 (of a corresponding one of struts 122).

Flow conditioner 140 is one example of fin-type flow conditioners 110 as described above with reference to FIGS. 3A-4. Flow conditioner 140 has one or more attachment regions 142 along its length (e.g., from a root portion to a tip portion). Attachment region 142 is a region where flow conditioner 140 is connected to frame 112 at one of struts 122. In some examples, attachment region 142 can be a single point. Attachment angle 148 is formed between fin longitudinal axis 150 of flow conditioner 140 and strut longitudinal axis 152 of a corresponding one of struts 122 to which flow conditioner 140 is attached. Attachment angle 148, as illustrated in FIG. 5A, is a position of flow conditioner 140 in a non-deflected or initial state. Flow conditioner 140 can be angled radially inward from frame 112 at attachment angle 148. In some examples, attachment angle 148 is ninety degrees or less. In other examples, attachment angle 148 is greater than ninety degrees. For example, attachment angle 148 can be selected so that flow conditioner 140 is angled away from other components of device 100 or so that flow conditioner 140 interacts optimally with blood flowing through or out of device 100. Deflection angle 149, as illustrated in FIG. 5B, is formed between fin longitudinal axis 150 and strut longitudinal axis 152 when flow conditioner 140 is deflected. Deflection angle 149 represents a change from attachment angle 148. Generally, deflection angle 149 is smaller than attachment angle 148. Attachment angle 148 and/or deflection angle 149 can be selected or calibrated to optimize the position of flow conditioner 140 based on a low heart rate or a high heart rate and low or high velocity blood flow.

Flow conditioner 140 is attached to frame 112 at attachment region 142, thereby forming joint 144 with a corresponding portion of frame 112. More specifically, joint 144 is formed between flow conditioner 140 and one of struts 122. Joint 144 can include any suitable attachment mechanism, such as a hinge, a flexible section of tissue or another material, a spring, etc. In some examples, joint 144 is a flexible joint that readily permits deflection of flow conditioner 140. In other examples, joint 144 is a rigid joint (e.g., if flow conditioner 140 is integrally formed with the corresponding strut 122).

Flow conditioner 140 can also be biased radially inwards from frame 112 by bias member 146, which can be a spring or other suitable feature for biasing flow conditioner 140. Bias member 146 is attached to one or more of struts 122. In some examples, joint 144 and bias member 146 are on a same one of struts 122. In other examples, joint 144 can be on a first one of struts 122 and bias member 146 can be attached to a second one of struts 122. In some such examples, the second one of struts 122 to which bias member 146 is attached can be adjacent to the first one of struts 122. In yet other examples, device 100 does not include bias member 146.

As illustrated in FIG. 5A, flow conditioner 140 is in a first, or non-deflected, position when there is low blood flow or when relatively lower velocity blood flows through device 100. The non-deflected position is represented by attachment angle 148. As illustrated in FIG. 5B, flow conditioner 140 deflects toward frame 112 when there is increased blood flow or when relatively higher velocity blood flows through device 100. The deflected flow conditioner 140 is at deflection angle 149, which can be a smaller angle compared to attachment angle 148. Additionally, bias member 146 will be compressed back toward frame 112.

The attachment angle 148 at joint 144 allows the initial positioning of flow conditioners 140 to be adjusted based on a desired hemodynamic effect. Moreover, the amount of disruption (or flow modification) caused by flow conditioner 140 on blood flowing through or out of device 100 can be calibrated at attachment angle 148 or by the incorporation of bias member 146. Specifically, the amount of deflection of flow conditioner 140 in response to increased blood flow (or increased velocity of blood flow) can be controlled by a tension of bias member 146. Bias member 146 can be adjusted to permit greater or lesser deflection of flow conditioner 140 based on a desired interaction between flow conditioner 140 and blood flowing through or out of device 100. Additionally, deflectable flow conditioners 140 can have increased compliance. These characteristics allow flow conditioner 140 to be flexibly implemented in a wide variety of implantable devices to cause different hemodynamic effects, and the positioning of flow conditioner 140 can be tuned for each implementation.

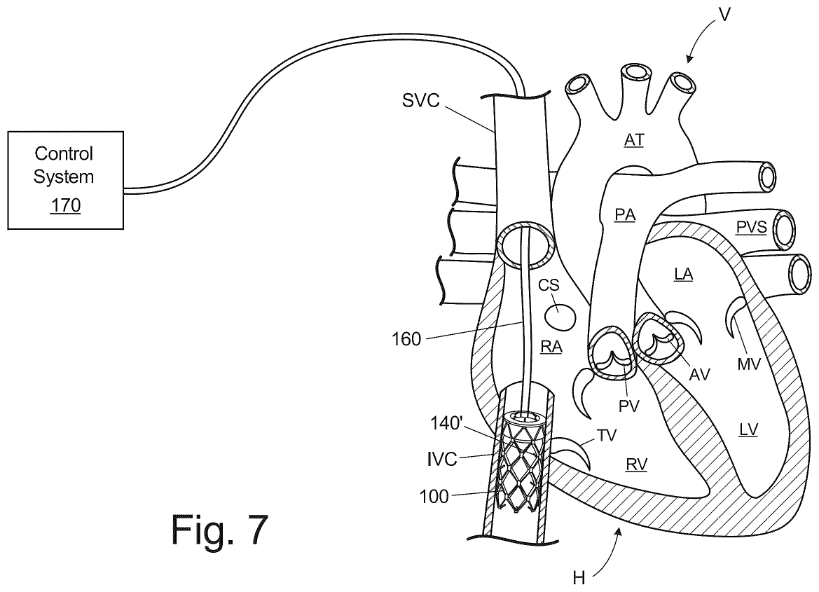

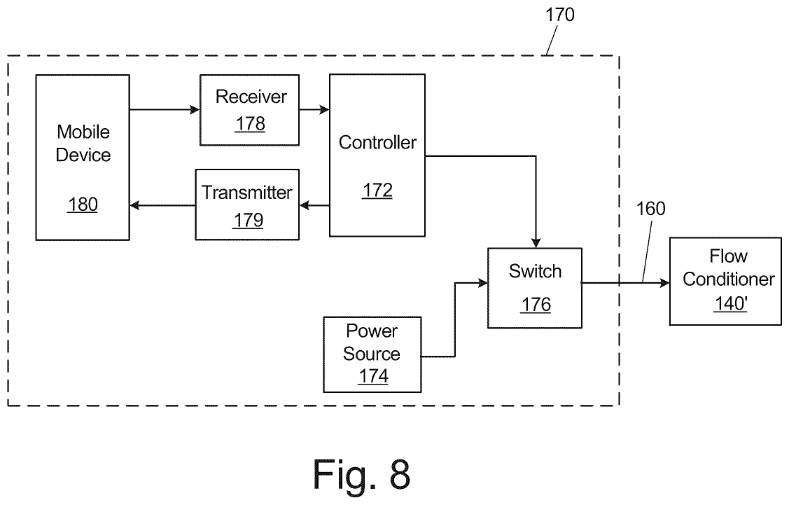

FIGS. 6A-8 will be described together. FIG. 6A is a schematic diagram illustrating connection of control components to actively controlled flow conditioner 140′. FIG. 6B is a schematic diagram illustrating electromechanical actuation of actively controlled flow conditioners 140′. FIG. 7 is a sectional view of heart H illustrating an example positioning of the control components for actively controlled flow conditioner 140′. FIG. 8 is a schematic diagram illustrating control system 170 for actively controlled flow conditioner 140′.

FIGS. 6A-6B show frame 112 (of cardiovascular implant device 100) including struts 122, flow conditioner 140′ including extension region 142′, electrical connectors 160, and control system 170. FIGS. 6A-6B also show extension angle 148′, adjusted angle 149′, fin longitudinal axis 150, and strut longitudinal axis 152 (of a corresponding one of struts 122). FIG. 7 shows cardiovascular implant device 100 including actively controlled flow conditioners 140′, electrical connectors 160, and control system 170. FIG. 7 also shows heart H, vasculature V, right atrium RA, right ventricle RV, left atrium LA, left ventricle LV, superior vena cava SVC, inferior vena cava IVC, tricuspid valve TV, pulmonary valve PV, pulmonary artery PA, pulmonary veins PVS, mitral valve MV, aortic valve AV, aorta AT, and coronary sinus CS. FIG. 8 shows actively controlled flow conditioner 140′, electrical connectors 160, and control system 170, which includes controller 172, power source 174, switch 176, receiver 178, transmitter 179, and mobile device 180.

Flow conditioner 140′ includes a similar structure and function as described above with respect to flow conditioners 110 (FIGS. 3A-4) and 140 (FIGS. 5A-5B), except flow conditioner 140′ is electromechanically actuated by control system 170.

Flow conditioner 140′ can be monolithically formed with one or more of struts 122 of frame 112. More specifically, flow conditioner 140′ and a portion of frame 112 to which flow conditioner 140′ is connected (one or more struts 122) can be monolithically formed of a shape-memory alloy, such as nitinol. In other examples, flow conditioner 140′ can be connected to frame 112 by an electrically controllable mechanism, such as a motorized hinge. Flow conditioner 140′ has one or more extension regions 142′ along its length (e.g., from a root portion to a tip portion). Extension region 142′ is a region where flow conditioner 140′ extends from frame 112 at one (or more) of struts 122. In some examples, flow conditioner 140′ extends monolithically from frame 112 at extension region 142′.

Extension angle 148′ is formed between fin longitudinal axis 150 of flow conditioner 140′ and strut longitudinal axis 152 of a corresponding one of struts 122 from which flow conditioner 140′ extends. Extension angle 148′, as illustrated in FIG. 6A, is a position of flow conditioner 140′ in an initial state (e.g., at body temperature). Flow conditioner 140′ can be angled radially inward from frame 112 at extension angle 148′. In some examples, extension angle 148′ is ninety degrees or less. In other examples, extension angle 148′ is greater than ninety degrees. For example, extension angle 148′ can be selected so that flow conditioner 140′ is angled away from other components of device 100 or so that flow conditioner 140 interacts optimally with blood flowing through or out of device 100. Moreover, the angle of flow conditioner 140′ is controllable during electromechanical actuation of flow conditioner 140′. In some examples, the angle of flow conditioner 140′ is controllable based on preset shapes of the shape-memory alloy that makes up at least the portion of frame 112 from which flow conditioner 140′ extends. Adjusted angle 149′ is formed between fin longitudinal axis 150 and strut longitudinal axis 152 when flow conditioner 140′ is electromechanically actuated. That is, in examples where flow conditioner 140′ and a corresponding portion of frame 112 are monolithically formed, different adjusted angles 149′ of flow conditioner 140′ can be preset and remembered by the shape-memory alloy when current is applied to flow conditioner 140′ to cause the shape-memory alloy to reach its transformation temperature. Adjusted angle 149′ represents a change from extension angle 148′. Generally, adjusted angle 149′ is smaller than extension angle 148′. Extension angle 148′ and/or adjusted angle 149′ can be selected or calibrated to optimize the position of flow conditioner 140′ based on a low heart rate or a high heart rate and low or high velocity blood flow.

Electrical connectors 160 are electrical connections between flow conditioner 140′ and control system 170. As shown in FIG. 7, electrical connectors 160 extend from device 100 through right atrium RA and through superior vena cava SVC. Electrical connectors 160 can generally be placed or fed through the right side of the cardiovascular system to avoid larger arteries, such as aorta AT, and to stay within veins, such as superior vena cava SVC and the subclavian vein (not shown). That is, although not shown in FIG. 7, electrical connectors 160 can extend from superior vena cava SVC through a portion of the thoracic vasculature (e.g., the subclavian vein) and out of the body via a puncture or incision to an externally located control system 170. In other examples, electrical connectors 160 can be placed in or along any vessels or chambers of heart H that are convenient with respect to the location of cardiovascular implant device 100. As shown in FIG. 8, electrical connectors 160 are connected to switch 176 of control system 170.

Control system 170 is a system of components for controlling (e.g., electromechanically actuating) flow conditioners 140′. Control system 170 can include wired or wireless connections between components. Moreover, all or some of components of control system 170 can be externally located outside the body. In one example, control system 170 can include receiver 178 and transmitter 179 in order to wirelessly communicate with mobile device 180 to receive and send signals for controlling flow conditioners 140′. In some examples, receiver 178 and transmitter 179 can be a transceiver. In other examples, control system 170 may not include receiver 178, transmitter 179, and mobile device 180, and instead controller 172 can directly implement predefined process instructions for controlling flow conditioners 140′. Alternatively, control system 170 may not include controller 172, and instead switch 176 can be manually activated to supply current from power source 174 to flow conditioners 140′.

Controller 172 is configured to implement process instructions for operational control of flow conditioners 140′. For instance, controller 172 can include one or more processors and computer-readable memory configured to implement functionality and/or process instructions for execution within control system 170. Examples of one or more processors can include, e.g., any one or more of a microprocessor, a controller, a digital signal processor (DSP), an application specific integrated circuit (ASIC), a field-programmable gate array (FPGA), or other equivalent discrete or integrated logic circuitry.

Computer-readable memory of controller 172 can be configured to store information used by controller 172 during operation of control system 170. Computer-readable memory, in some examples, is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). In some examples, the computer-readable memory is used to store program instructions for execution by the one or more processors. Computer-readable memory, in one example, is used by software or applications running on controller 172 to temporarily store information during program execution. Computer-readable memory can include volatile and non-volatile memories. Examples of volatile memories can include, e.g., random access memories (RAM), dynamic random access memories (DRAM), static random access memories (SRAM), and other forms of volatile memories. Examples of non-volatile storage elements can include, for example, magnetic hard discs, optical discs, flash memories, or forms of electrically programmable memories (EPROM) or electrically erasable and programmable (EEPROM) memories.

Examples of controller 172 can include any one or more of microcontrollers or other computers. Controller 172 can be configured to communicate with any one or more of the components of control system 170, including: switch 176, receiver 178, and transmitter 179. Although the example of FIG. 8 illustrates controller 172 as operatively coupled to other components of control system 170, other examples can include a dedicated device where controller 172 is integrated with switch 176, receiver 178, and transmitter 179 to control flow conditioners 140′.

Power source 174 supplies current to flow conditioner 140′ via electrical connectors 160. In some examples, power source 174 can be a battery. Switch 176 is between power source 174 and flow conditioner 140′. Switch 176 can be activated (or turned on) such that current can flow from power source 174 to flow conditioner 140′. Switch 176 can also be deactivated (or turned off) such that current does not flow from power source 174 to flow conditioner 140′. Switch 176 can be activated or deactivated manually or by a control signal from controller 172. For example, switch 176 can be a metal-oxide semiconductor field-effect transistor (MOSFET) or any other electrical switch.

Controller 172 is connected to or includes receiver 178 and transmitter 179 (or a transceiver rather than separate transmitter and receiver) for sending and receiving wireless signals. For example, receiver 178 can receive a Bluetooth Low Energy (BLE) signal. In other examples, receiver 178 can receive a Wi-Fi signal. In yet other examples, receiver 178 can be a receiver for any suitable wireless signal type. Similarly, transmitter 179 can be a transmitter for any suitable wireless signal type. In some examples, receiver 178 can receive signals from mobile device 180, and transmitter 179 can transmit signals to mobile device 180.

Mobile device 180 is an access point for remotely controlling flow conditioners 140′. For example, mobile device 180 can be a cell phone, tablet, or other device capable of sending a wireless signal to receiver 178 to communicate with controller 172. Mobile device 180 can include a user interface (UI) for displaying control options for control system 170 to a user, such as a physician. Mobile device 180 can include a display and/or other UI elements (e.g., keyboard, buttons, monitor, graphical control elements presented at a touch-sensitive display, or other UI elements). In some examples, mobile device includes a graphical user interface (GUI) that includes graphical representations of control options for control system 170, such as graphical representations of a button for activating switch 176.

In operation of control system 170, switch 176 is activated so that current can flow from power source 174 to flow conditioner 140′ along electrical connectors 160. Switch 176 can be manually activated or can be activated based on a signal from controller 172. Controller 172 can send signals based on predefined instructions, such as configurations, stored thereon or can receive a signal from mobile device 180 via receiver 178. The supplied current causes flow conditioner 140′ to deflect to adjusted angle 149′. In some examples, flow conditioner 140′ that is formed of a shape-memory alloy reaches its transformation temperature and shifts to a preset shape that coincides with the transformation temperature. That is, as illustrated in FIG. 6B, flow conditioner 140′ will deflect from extension angle 148′ to adjusted angle 149′ based on preset shapes of the shape-memory alloy. In other examples, the supplied current activates an electrically controllable hinge or other active mechanism at extension region 142′. On the other hand, when switch 176 is deactivated, current is no longer supplied to flow conditioner 140′, and flow conditioner 140′ will return to its initial position at extension angle 148′, as illustrated in FIG. 6A.

Control system 170 allows the positioning of flow conditioner 140′ to be actively controlled or adjusted once device 100 has been implanted in the cardiovascular system. That is, the amount of disruption (or flow modification) caused by flow conditioners 140′ on blood flowing through or out of device 100 can be actively calibrated by changing extension angle 148′ to adjusted angle 149′ in response to supplied current from power source 174. In this way, a user such as a physician or a patient can adjust the position of flow conditioner 140′ based on changed conditions without having to directly access flow conditioner 140′ (e.g., via a surgical procedure or other route). For example, in response to a worsening heart disease, the position of flow conditioner 140′ could be adjusted to have a more significant effect on hemodynamics. Further, control system 170 using mobile device 180 allows remote control and adjustment of the positioning of flow conditioner 140′, which may streamline a procedure for adjusting the positioning of flow conditioner 140′ or provide a user-friendly alternative option for adjusting the positioning of flow conditioner 140′. Forming flow conditioner 140′ and a corresponding portion of frame 112 of a shape-memory alloy also allows for active control of flow conditioner 140′ by a relatively minor modification to the structure of device 100.

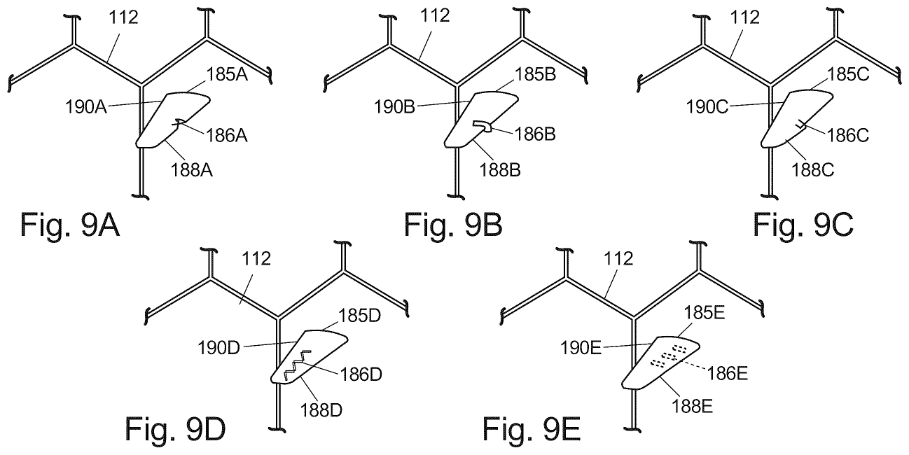

FIGS. 9A-9E will be described together. FIGS. 9A-9E are enlarged partial perspective views of frame 112 of cardiovascular implant device 100 illustrating several variations of fin-type flow conditioners. The fin-type flow conditioners described herein can take a number of different forms. Five examples are provided with reference to FIGS. 9A-9E. These examples are not intended to be limiting, and other examples are possible. FIG. 9A shows flow conditioner 185A attached to a portion of frame 112 and including flow microfeatures 186A. Flow conditioner 185A further includes leading edge 188A and trailing edge 190A. FIG. 9B shows flow conditioner 185B attached to a portion of frame 112 and including flow microfeatures 186B. Flow conditioner 185B further includes leading edge 188B and trailing edge 190B. FIG. 9C shows flow conditioner 185C attached to a portion of frame 112 and including flow microfeatures 186C. Flow conditioner 185C further includes leading edge 188C and trailing edge 190C. FIG. 9D shows flow conditioner 185D attached to a portion of frame 112 and including flow microfeatures 186D. Flow conditioner 185D further includes leading edge 188D and trailing edge 190D. FIG. 9E shows flow conditioner 185E attached to a portion of frame 112 and including flow microfeatures 186E. Flow conditioner 185E further includes leading edge 188E and trailing edge 190E.

Each of flow conditioners 185A, 185B, 185C, 185D, and 185E can one of fin-type flow conditioners 110 shown in FIGS. 3A-4, fin-type flow conditioner 140 shown in FIGS. 5A-5B, or fin-type flow conditioner 140′ shown in FIGS. 6A-8. Specifically, each of flow conditioners 185A, 185B, 185C, 185D, and 185E can be an airfoil. Flow conditioner 185A extends between leading edge 188A and trailing edge 190A, flow conditioner 185B extends between leading edge 188B and trailing edge 190B, flow conditioner 185C extends between leading edge 188C and trailing edge 190C, flow conditioner 185D extends between leading edge 188D and trailing edge 190D, and flow conditioner 185E extends between leading edge 188E and trailing edge 190E.

Flow conditioner 185A includes flow microfeature 186A, which is a leading edge notch. Flow conditioner 185B includes flow microfeature 186B, which is a boundary layer fence. Flow conditioner 185C includes flow microfeature 186C, which is a leading edge dogtooth. Flow conditioner 185D includes flow microfeature 186D, which is a group of vortex generators. Flow conditioner 185E includes flow microfeature 186E, which is a group of vortilons (shown in FIG. 9E with illusion lines through flow conditioner 185E from an opposite side of flow conditioner 185E with respect to the viewing plane). For example, flow microfeatures 186A, 186B, 186C, 186D, and 186E can be located at or proximal to the respective leading edge 188A, 188B, 188C, 188D, and 188E of the corresponding flow conditioner 185A, 185B, 185C, 185D, and 185E. As shown in FIGS. 9A-9C, flow microfeatures 186A, 186B, and 186C are located along respective leading edges 188A, 188B, and 188C. As shown in FIGS. 9D-9E, flow microfeatures 186D and 186E are located proximal to respective leading edges 188D and 188E. Other fin-type flow conditioners can include a combination of the flow microfeatures described herein. Further, device 100 can include any combination of flow conditioners 185A, 185B, 185C, 185D, and 185E and flow microfeatures 186A, 186B, 186C, 186D, and 186E arranged throughout frame 112 in any suitable pattern or organization, depending on the desired hemodynamic characteristics.

Flow microfeatures 186A, 186B, 186C, 186D, and 186E interact with blood flowing through or out of device 100 as it reaches flow conditioners 185A, 185B, 185C, 185D, and 185E. Each of flow microfeatures 186A, 186B, 186C, 186D, and 186E can produce characteristic flow effects on the flow of blood through or out of device 100.

Flow conditioners 185A, 185B, 185C, 185D, and 185E including flow microfeatures 186A, 186B, 186C, 186D, and 186E provide a wider range of options and greater flexibility for designing cardiovascular implant devices to modify hemodynamic characteristics for producing desired hemodynamic effects on blood flowing through or out of the device. Incorporating flow microfeatures 186A, 186B, 186C, 186D, and 186E or combinations of flow microfeatures 186A, 186B, 186C, 186D, and 186E can result in finer or more granular control of hemodynamic effects.

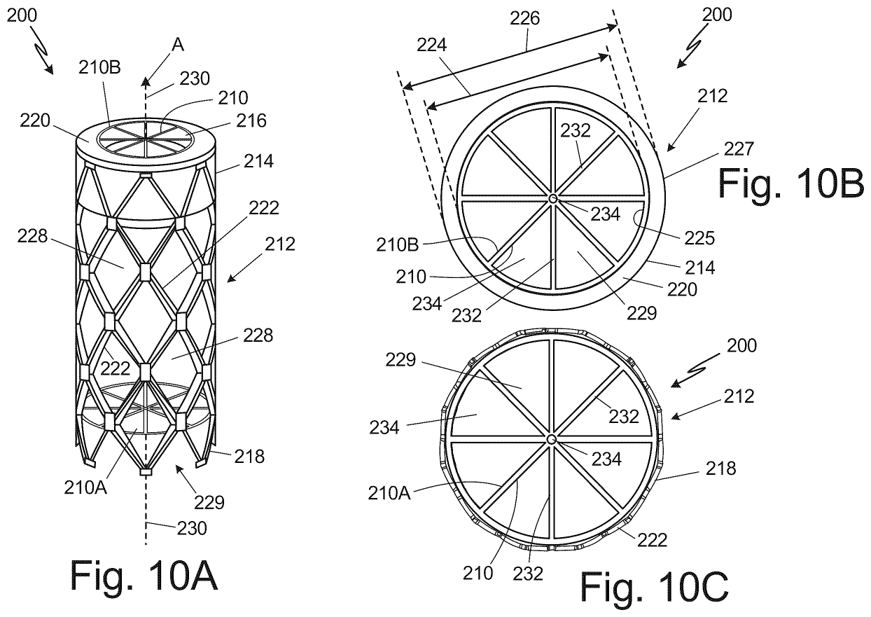

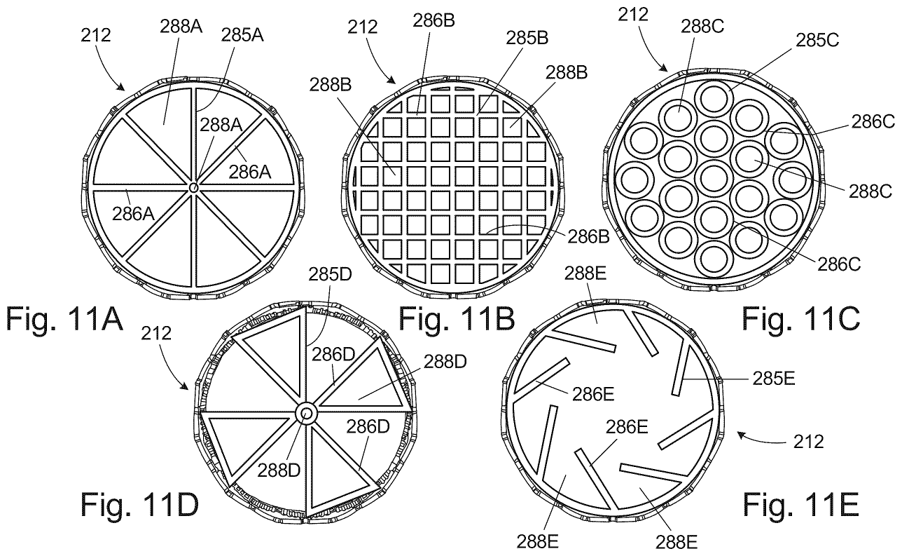

### DEVICE 200 (FIGS. 10A-11E)

FIGS. 10A-10C will be described together. FIG. 10A is a perspective view of cardiovascular implant device 200 including plate-type flow conditioners 210. FIG. 10B is a top view of cardiovascular implant device 200 including plate-type flow conditioners 210. FIG. 10C is a bottom view of cardiovascular implant device 200 including plate-type flow conditioners 210. As illustrated in FIGS. 10A-10C, cardiovascular implant device 200 includes plate-type flow conditioners 210, including flow conditioner 210A and 210B (which will be referred to collectively herein by the shared reference number), frame 212, cover 214, valve seat 216, inflow end 218, and outflow end 220. Frame 212 includes struts 222, inner diameter 224, inner surface 225, outer diameter 226, and outer surface 227, and defines openings 228, central flow path 229, and flow axis 230. Flow conditioners 210 include walls 232 and define flow passages 234 therein.

Cardiovascular implant device 200 includes a similar structure and function to cardiovascular implant device 100 described above, except device 200 includes plate-type flow conditioners 210 instead of fin-type flow conditioners (e.g., flow conditioners 110).

Flow conditioners 210 are flow plates or plate-type flow conditioners. Each individual one of flow conditioners 210 can also be referred to as a flow conditioner feature. Flow conditioners 210 can be flattened or cylindrical projections from frame 212. More specifically, flow conditioners 210 are connected to frame 212 at corresponding ones of struts 222. In some examples, flow conditioners 210 are attached to multiple struts 222 along portions of frame 212. In some examples, flow conditioners 210 are connected circumferentially at several locations along inner surface 225 of frame 212. In some examples, flow conditioners 210 are continuously formed with inner surface 225 of frame 212. Flow conditioners 210 are attached by an attachment mechanism or monolithically formed with a portion of frame 212. Each plate-type flow conditioner 210 can be a single part rather than a plurality of individual fin-type flow conditioners.

In general, flow conditioners 210 can take a number of different forms (i.e., shapes, sizes, etc.). Physical dimensions (e.g., length, width, shape, cross-sectional shape, etc.) of flow conditioners 210 can be configured to prevent flow conditioners 210 from interfering (or contacting) other parts of device 200 or adjacent tissue walls. In other examples, components of device 200 (e.g., cover 214) can be designed to fit around flow conditioners 210 or to permit flow conditioners 210 to pass through. The physical dimension of flow conditioners 210 can further be configured to allow flow conditioners 210 to collapse and expand with expandable frame 212 (e.g., to fit within a delivery catheter). The physical dimensions of flow conditioners 210 can further be configured to prevent flow conditioners 210 from occluding a vessel of chamber of heart H in which device 200 is implanted. That is, a length and/or width of flow conditioners 210 can be relatively short enough so that flow conditioners 210 do not protrude from device 200 and extend fully across a vessel or chamber of heart H and block blood flow. The physical dimensions of flow conditioners 210 can further be configured to modify a hemodynamic characteristic (e.g., helicity, vorticity, velocity, turbulence, flow direction, etc.) of blood flowing through or out of device 200 in a particular manner.

One or more flow conditioners 210 can be positioned in any suitable arrangement with respect to frame 212 of device 200. In the example shown in FIG. 10A, device 200 includes two flow conditioners: flow conditioner 210A and flow conditioner 210B. Other examples can include any number of flow conditioners 210. Flow conditioners 210 are positioned to span across a portion of central flow path 229 (defined by frame 212) such that flow conditioners 210 intersect flow axis 230 through frame 212. In some examples, flow conditioners 210 can be located adjacent to inflow end 218 (flow conditioner 210A as shown in FIG. 10C) and/or outflow end 220 (flow conditioner 210B as shown in FIG. 10B). In some examples, flow conditioners 210 are connected to ones of struts 222 that form a first row of openings 228 adjacent to outflow end 220. Device 200 can include any number of flow conditioners 210 in any one or more of the foregoing locations. Locations of flow conditioners 210 can be configured to prevent flow conditioners 210 from interfering with other parts of device 200 or adjacent tissue walls. In other examples, components of device 200 (e.g., cover 214) can be designed to fit around flow conditioners 210 or to permit flow conditioners 210 to pass through. The locations of flow conditioners 210 can further be configured to allow flow conditioners 210 to collapse and expand with expandable frame 212 (e.g., to fit within a delivery catheter). The locations of flow conditioners 210 can further be configured to prevent flow conditioners 210 from occluding a vessel or chamber of heart H in which device 200 is implanted. The locations of flow conditioners 210 can further be configured to modify a hemodynamic characteristic (e.g., helicity, vorticity, velocity, turbulence, flow direction, etc.) of blood flowing through or out of device 200 in a particular manner.

Each flow conditioner 210 includes walls 232 that define flow passages 234 therein. Flow passages 234 extend through a corresponding one of flow conditioners 210 such that blood flowing along flow axis 230 through central flow path 229 within device 200 can pass through the corresponding flow conditioner 210. That is, flow passages 234 extend from an upstream side to a downstream side of the corresponding flow conditioner 210. The form of each flow conditioner 210 can depend largely on the configuration of flow passages 234. Flow passages are bounded, at least partially, by walls 232. In some examples, flow passages 234 are closed channels that are surrounded by walls 232 (as shown in FIGS. 10A-10C). In other examples, flow passages 234 can be continuous with adjacent flow passages 234 by gaps or spaces between walls 232. Individual walls 232 can have any height (as measured with respect to flow axis 230). In some examples, walls 232 can have a same height to form a flat upstream and/or downstream surface of flow conditioner 210. In other examples, walls 232 can protrude such that the upstream and/or downstream surface of flow conditioner 210 is not flat. Flow passages 234 can take a number of different forms (i.e., shapes, sizes, curvatures, etc.). The example illustrated in FIGS. 10A-10C includes eight wedge shaped flow passages 234 and one central circular flow passage 234 (when viewed perpendicular to an upstream or downstream side of flow conditioner 210). Other examples can include any combination of shapes and sizes of flow passages 234 throughout flow conditioners 210.

Once device 200 is implanted in the cardiovascular system, circulating blood passes through device 200. As blood flows, into, through, and out of device 200 along flow axis 230, the blood flows through flow passages 234 of flow conditioners 210. Flow conditioners 210 interact with the blood flow to modify or affect a hemodynamic characteristic (e.g., helicity, vorticity, velocity, turbulence, flow direction, etc.) of the flow. Flow conditioners 210 can interact with blood flowing through or out of device 200 by adding flow resistance and/or changing the direction of the blood flow to prevent reversal of blood flow. For example, flow conditioners 210 may increase or decrease vorticity or helicity of the flow. In some examples, flow conditioners 210 may cause the flow to be smoother (decrease the turbulence). In other examples, flow conditioners 210 can increase turbulence in the flow. In some examples, flow conditioners 210 can change a flow direction of the flow. In some examples, flow conditioners 210 can align the flow with a natural vortical flow pattern of blood through a vessel or chamber of heart H, such as the left-sided flow vortex in left atrium LA or the right-sided flow vortex in right atrium RA. In other examples, flow conditioners 210 can align the flow with a natural helical flow pattern of blood though a vessel or chamber of heart H, such as helical flow in coronary sinus CS. Flow conditioners 210 that are located at inflow end 218 and/or outflow end 220 of device 200 can create helical flow patterns near an adjacent vessel or chamber wall to force blood to flow back towards the center of the vessel or chamber in a same helical direction, thereby producing forward movement of the blood. More generally, flow conditioners 210 adjacent to inflow end 218 can modify a hemodynamic characteristic of blood flowing through frame 212, and flow conditioners 210 adjacent to outflow end 220 can modify a hemodynamic characteristic of blood flowing out of frame 212.

Like device 100 described above, cardiovascular implant device 200, including flow conditioners 210, can produce hemodynamic effects to minimize disruption to or enhance the natural flow patterns in heart H, such as the left-sided flow vortex in left atrium LA, the right-sided flow vortex in right atrium RA, and/or helical flow in coronary sinus CS. Flow conditioners 210 can modify hemodynamic characteristics of blood flowing through or out of device 200 such that (a) any disruptions to the natural flow patterns that would be caused by an implantable device are minimized; (b) reduced flow due to a pathophysiology or other cause is mitigated; and/or (c) baseline flow is enhanced. As a result, device 200 can maintain kinetic energy of the cardiovascular blood flow, which in turn reduces the cardiac work needed and improves cardiac efficiency. These hemodynamic effects can potentially improve patient outcomes after receiving cardiovascular implant device 200 because device 200 can be more effective and potentially safer. At the same time, flow conditioners 210 can be incorporated relatively easily into device 200 in many configurations, so different variations of device 200 can be optimized for use in many different scenarios (e.g., for many different patient conditions).

FIGS. 11A-11E will be described together. FIGS. 11A-11E are perspective views of cardiovascular implant device 200 illustrating several variations of plate-type flow conditioners. The plate-type flow conditioners described herein can take a number of different forms. Five examples are provided with reference to FIGS. 11A-11E. These examples are not intended to be limiting, and other examples are possible. FIG. 11A shows flow conditioner 285A attached to a portion of frame 212 and including walls 286A, which define flow passages 288A therein. FIG. 11B shows flow conditioner 285B attached to a portion of frame 212 and including walls 286B, which define flow passages 288B therein. FIG. 11C shows flow conditioner 285C attached to a portion of frame 212 and including walls 286C, which define flow passages 288C therein. FIG. 11D shows flow conditioner 285D attached to a portion of frame 212 and including walls 286D, which define flow passages 288D therein. FIG. 11E shows flow conditioner 285E attached to a portion of frame 212 and including walls 286E, which define flow passages 288E therein.

Each of flow conditioners 285A, 285B, 285C, 285D, and 285E can be plate-type flow conditioners 210 shown in FIGS. 10A-10C. Flow conditioner 285A includes walls 286A, which take the form of connected fins that extend across flow conditioner 285A in a wheel shape. Accordingly, walls 286A form flow passages 288A that have wedge shaped cross sections. Walls 286A also form a central flow passage 288A that has a circular cross section. Flow conditioner 285A is the example of flow conditioners 210 that is depicted in FIGS. 10A-10C. Flow conditioner 285B includes walls 286B, which take the form of a grid or lattice of flow passages 288B (such as a Zanker-type flow plate). Flow conditioner 285C includes walls 286C, which take the form of connected tubes surrounding flow passages 288C. Flow conditioner 285D includes walls 286D, which take the form of folded vanes in a wheel shape similar to flow conditioner 285A, except some flow passages 288D formed by walls 286D are bounded by an additional wall 286D rather than frame 212. Accordingly, walls 286D form flow passages 288D that have wedge shaped cross sections. Walls 286D also form a central flow passage 288D that has a circular cross section. Flow conditioner 285E includes walls 286E, which take the form of angled tabs that form flow passages 288E therebetween. Flow passages 288E are continuous with each other centrally. Other plate-type flow conditioners can include a combination of the walls and flow passages described herein. Further, device 200 can include any combination of flow conditioners 285A, 285B, 285C, 285D, and 285E arranged along frame 212 in any suitable pattern or organization, depending on the desired hemodynamic characteristics.

Flow conditioners 285A, 285B, 285C, 285D, and 285E interact with blood flowing through or out of device 200 as it passes through the corresponding flow passages 288A, 288B, 288C, 288D, and 288E. Flow conditioners 285A, 285B, 285C, 285D, and 285E, each having different respective types or arrangements of walls 286A, 286B, 286C, 286D, and 286E and flow passages 288A, 288B, 288C, 288D, and 288E, can produce characteristic flow effects on the flow of blood through or out of device 200.

Flow conditioners 285A, 285B, 285C, 285D, and 285E including walls 286A, 286B, 286C, 286D, and 286E and flow passages 288A, 288B, 288C, 288D, and 288E provide a wider range of options and greater flexibility for designing cardiovascular implant devices to modify hemodynamic characteristics for producing desired hemodynamic effects on blood flowing through or out of the device. Incorporating flow conditioners 285A, 285B, 285C, 285D, and 285E including walls 286A, 286B, 286C, 286D, and 286E and flow passages 288A, 288B, 288C, 288D, and 288E or combinations of these can result in finer or more granular control of hemodynamic effects.

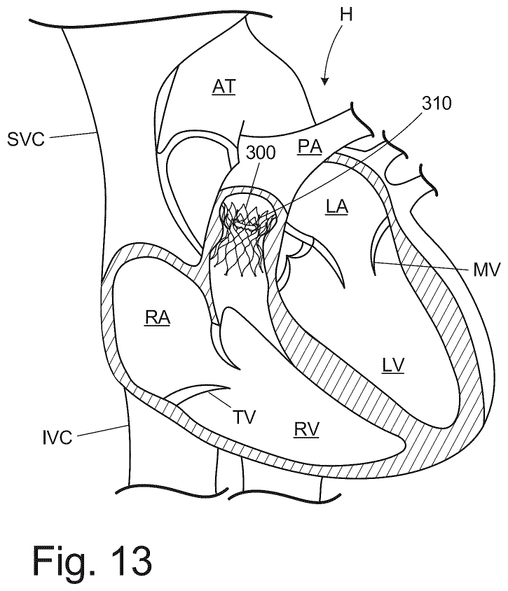

### DEVICE 300 (FIGS. 12-13)

FIGS. 12 and 13 will be described together. FIG. 12 is a perspective view of cardiovascular implant device 300 including fin-type flow conditioners 310. FIG. 13 is a sectional view of heart H illustrating an example positioning at a valve site of cardiovascular implant device 300 including fin-type flow conditioners 310. As illustrated in FIGS. 12-13, cardiovascular implant device 300 includes fin-type flow conditioners 310, frame 312, cover 314, valve seat 316, valve 317, inflow end 318, and outflow end 320. Frame 312 includes struts 322, inner diameter 324, inner surface 325, outer diameter 326, and outer surface 327, and defines openings 328, central flow path 329, and flow axis 330. FIG. 13 also shows device 300, heart H, right atrium RA, right ventricle RV, left atrium LA, left ventricle LV, superior vena cava SVC, inferior vena cava IVC, tricuspid valve TV, pulmonary valve PV, pulmonary artery PA, mitral valve MV, aortic valve AV, and aorta AT.

Cardiovascular implant device 300 includes a similar structure and function to cardiovascular implant device 100 described above, except device 300 is located at a valve site rather than a non-valve site. For example, FIG. 13 shows device 300 is positioned in pulmonary artery PA at the site of pulmonary valve PV such that inflow end 318 is facing right ventricle RV and outflow end 320 is within pulmonary artery PA. In other examples, device 300 can be located at aortic valve AV, mitral valve MV, or other valves. Cardiovascular implant device 300 also includes various minor structural variations compared to device 100. For example, frame 312 has a bi-directionally flared or generally hourglass shaped profile rather than a regular cylindrical shape. Valve seat 316 is located centrally along a longitudinal axis (e.g., flow axis 330) through cardiovascular implant device 300. Cardiovascular implant device 300 is also depicted in FIGS. 12 and 13 as including valve 317 supported in valve seat 316. Compared to cover 114 for device 100 as shown in FIG. 3A, cover 314 extends over a greater portion of frame 312. Moreover, cover 314 extends from inflow end 318 towards outflow end 320 but outflow end 320 is not covered by cover 314. It should be understood that, among other variations described above with reference to device 100, other examples of cardiovascular implant devices can include a wide variety of frame shapes and sizes and positions of valve seats and covers.

Fin-type flow conditioners 310 can generally include the same structure and function as fin-type flow conditioners 110 shown in FIGS. 3A-4), fin-type flow conditioner 140 shown in FIGS. 5A-5), fin-type flow conditioner 140′ shown in FIGS. 6A-8, and fin-type flow conditioners 185A-185E shown in FIGS. 9A-9E. One or more flow conditioners 310 can be positioned in any suitable arrangement with respect to frame 312 of device 300. In some examples, flow conditioners 310 can be arranged about a circumference of inner surface 325 of frame 312 that is defined by inner diameter 324. In some examples, flow conditioners 310 can be located adjacent inflow end 318 and/or outflow end 320. In some examples, flow conditioners 310 are connected to ones of struts 322 that form a first row of openings 328 that is adjacent to outflow end 320. Device 300 can include any number of flow conditioners 310 in any one or more of the foregoing locations. Locations of flow conditioners 310 can be configured to prevent flow conditioners 310 from interfering with other parts of device 300 or adjacent tissue walls. In other examples, components of device 300 (e.g., cover 314) can be designed to fit around flow conditioners 310 or to permit flow conditioners 310 to pass through. The locations of flow conditioners 310 can further be configured to allow flow conditioners 310 to collapse and expand with expandable frame 312 (e.g., to fit within a delivery catheter). The locations of flow conditioners 310 can further be configured to prevent flow conditioners 310 from occluding a vessel of chamber of heart H in which device 300 is implanted. The locations of flow conditioners 310 can further be configured to modify a hemodynamic characteristic (e.g., helicity, vorticity, velocity, turbulence, flow direction, etc.) of blood flowing through or out of device 300 in a particular manner.

Once device 300 is implanted in cardiovascular system (e.g., in pulmonary artery PA as shown in FIG. 13), circulating blood passes through device 300. As blood flows into, through, and out of device 300 along flow axis 330, flow conditioners 310 interact with the blood flow to modify or affect a hemodynamic characteristic (e.g., helicity, vorticity, velocity, turbulence, flow direction, etc.) of the flow. Flow conditioners 310 can interact with blood flowing through or out of device 300 by adding flow resistance and/or changing the direction of the blood flow to prevent reversal of blood flow. For example, flow conditioners 310 may increase or decrease vorticity or helicity of the flow. In some examples, flow conditioners 310 may cause the flow to be smoother (decrease the turbulence). In other examples, flow conditioners 310 can increase turbulence in the flow. In some examples, flow conditioners 310 can change a flow direction of the flow. In some examples, flow conditioners 310 can align the flow with a natural vortical flow pattern of blood through a vessel or chamber of heart H, such as the left-sided flow vortex in left atrium LA or the right-sided flow vortex in right atrium RA. In other examples, flow conditioners 310 can align the flow with a natural helical flow pattern of blood through a vessel or chamber of heart H, such as helical flow in coronary sinus CS. Flow conditioners 310 that are located circumferentially at inflow end 318 and/or outflow end 320 of device 300 can create helical flow patterns near an adjacent vessel or chamber wall to force blood to flow back towards the center of the vessel or chamber in a same helical direction, thereby producing forward movement of the blood. More generally, flow conditioners 310 adjacent to inflow end 318 can modify a hemodynamic characteristic of blood flowing through frame 312, and flow conditioners 310 adjacent to outflow end 320 can modify a hemodynamic characteristic of blood flowing out of frame 312.

In addition to the benefits described above with respect to device 100, device 300, according to techniques of this disclosure, allows for modifying hemodynamic characteristics at or adjacent the site of a native heart valve that has been replaced. Despite being in a location where a native valve was previously (and, therefore, the location of a natural flow path regulator), an implantable device may still disrupt natural flow patterns due to factors such as the size of the device, the position of the device, any contact between the device and nearby tissue, or other factors. Flow conditioners 310 incorporated on device 300 can produce hemodynamic effects to minimize disruption to or enhance the natural flow patterns at or adjacent to a valve site.

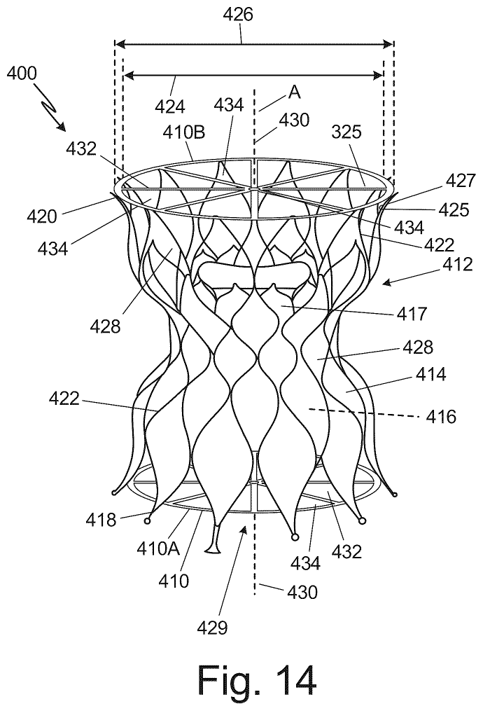

### DEVICE 400 (FIG. 14)

FIG. 14 is a perspective view of cardiovascular implant device 400 including plate-type flow conditioner 410. As illustrated in FIG. 14, cardiovascular implant device 400 includes plate-type flow conditioners 410, including flow conditioner 410A and 410B (which will be referred to collectively herein by the shared reference number), frame 412, cover 414, valve seat 416, valve 417, inflow end 418, and outflow end 420. Frame 412 includes struts 422, inner diameter 424, inner surface 425, outer diameter 426, and outer surface 427, and defines openings 428, central flow path 429, and flow axis 430. Flow conditioners 410 include walls 432 and define flow passages 434 therein.

Cardiovascular implant device 400 includes a similar structure and function to cardiovascular implant device 300 described above, except device 400 includes plate-type flow conditioners 410 instead of fin-type flow conditioners (e.g., flow conditioners 310). Further, plate-type flow conditioners 410 can generally include the same structure and function as plate-type flow conditioners 210 shown in FIGS. 10A-10C and plate-type flow conditioners 285A-285E shown in FIGS. 11A-11E. One or more flow conditioners 410 can be positioned in any suitable arrangement with respect to frame 412 of device 400. In the example shown in FIG. 14, device 400 includes two flow conditioners: flow conditioner 410A and flow conditioner 410B. Other examples can include any number of flow conditioners 410. Flow conditioners 410 are positioned to span across a portion of central flow path 429 (defined by frame 412) such that flow conditioners 410 intersect flow axis 430 through frame 412. In some examples, flow conditioners 410 can be located adjacent inflow end 418 (flow conditioner 410A) and/or outflow end 420 (flow conditioner 410B). In some examples, flow conditioners 410 are connected to ones of struts 422 that form a first row of openings 428 that is adjacent to outflow end 420. Device 400 can include any number of flow conditioners 410 in any one or more of the foregoing locations. Locations of flow conditioners 410 can be configured to prevent flow conditioners 410 from interfering with other parts of device 400 or adjacent tissue walls. In other examples, components of device 400 (e.g., cover 414) can be designed to fit around flow conditioners 410 or to permit flow conditioners 410 to pass through. The locations of flow conditioners 410 can further be configured to allow flow conditioners 410 to collapse and expand with expandable frame 412 (e.g., to fit within a delivery catheter). The locations of flow conditioners 410 can further be configured to prevent flow conditioners 410 from occluding a vessel of chamber of heart H in which device 400 is implanted. The locations of flow conditioners 410 can further be configured to modify a hemodynamic characteristic (e.g., helicity, vorticity, velocity, turbulence, flow direction, etc.) of blood flowing through or out of device 400 in a particular manner.

Once device 400 is implanted in the cardiovascular system, circulating blood passes through device 400. As blood flows, into, through, and out of device 400 along flow axis 430, the blood flows through flow passages 434 of flow conditioners 410. Flow conditioners 410 interact with the blood flow to modify or affect a hemodynamic characteristic (e.g., helicity, vorticity, velocity, turbulence, flow direction, etc.) of the flow. Flow conditioners 410 can interact with blood flowing through or out of device 400 by adding flow resistance and/or changing the direction of the blood flow to prevent reversal of blood flow. For example, flow conditioners 410 may increase or decrease vorticity or helicity of the flow. In some examples, flow conditioners 410 may cause the flow to be smoother (decrease the turbulence). In other examples, flow conditioners 410 can increase turbulence in the flow. In some examples, flow conditioners 410 can change a flow direction of the flow. In some examples, flow conditioners 410 can align the flow with a natural vortical flow pattern of blood through a vessel or chamber of heart H, such as the left-sided flow vortex in left atrium LA or the right-sided flow vortex in right atrium RA. In other examples, flow conditioners 410 can align the flow with a natural helical flow pattern of blood through a vessel or chamber of heart H, such as helical flow in coronary sinus CS. Flow conditioners 410 that are located at inflow end 418 and/or outflow end 420 of device 400 can create helical flow patterns near an adjacent vessel or chamber wall to force blood to flow back towards the center of the vessel or chamber in a same helical direction, thereby producing forward movement of the blood. More generally, flow conditioners 410 adjacent to inflow end 418 can modify a hemodynamic characteristic of blood flowing through frame 412, and flow conditioners 410 adjacent to outflow end 420 can modify a hemodynamic characteristic of blood flowing out of frame 412.

In addition to the benefits described above with respect to device 200, device 400, according to techniques of this disclosure, allows for modifying hemodynamic characteristics at or adjacent the site of a native heart valve that has been replaced. Despite being in a location where a native valve was previously (and, therefore, the location of a natural flow path regulator), an implantable device may still disrupt natural flow patterns due to factors such as the size of the device, the position of the device, any contact between the device and nearby tissue, or other factors. Flow conditioners 410 incorporated on device 400 can produce hemodynamic effects to minimize disruption to or enhance the natural flow patterns at or adjacent to a valve site.

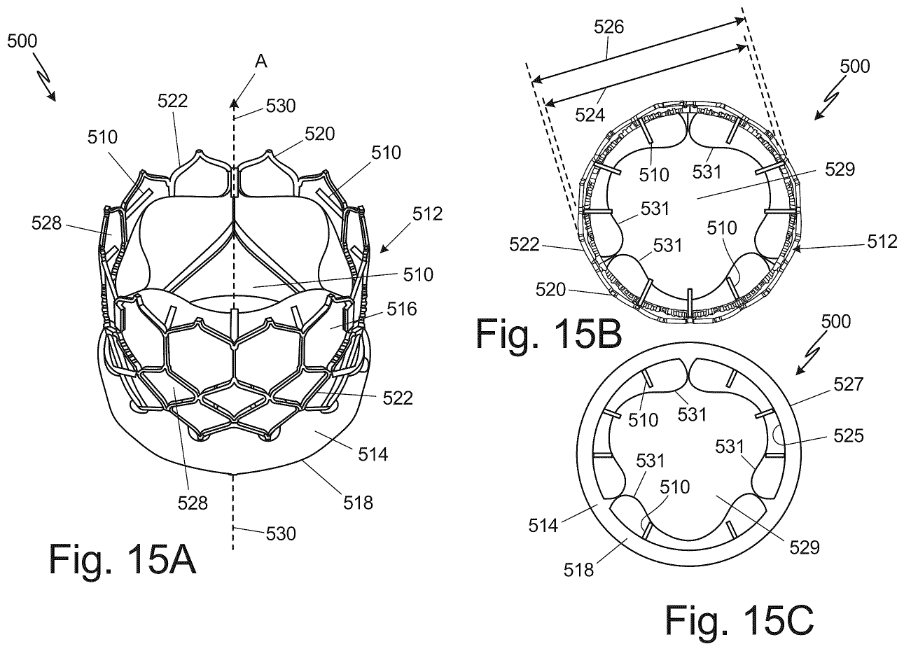

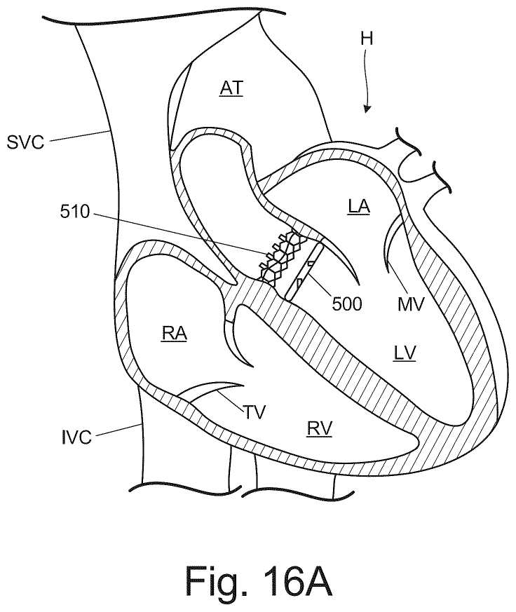

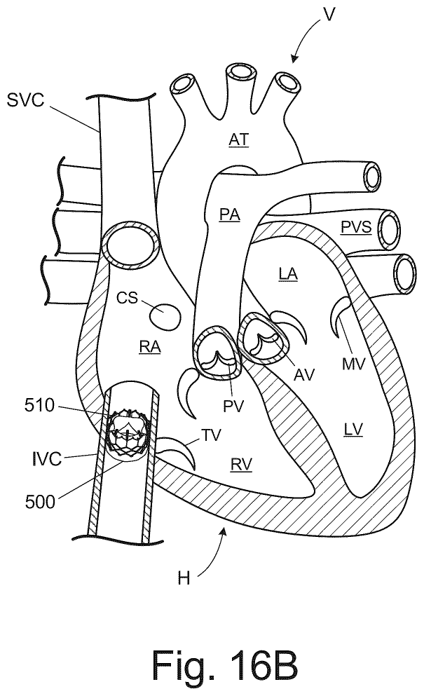

### DEVICE 500 (FIGS. 15A-16B)

FIGS. 15A-16B will be described together. FIG. 15A is a perspective view of cardiovascular implant device 500 including fin-type flow conditioners 510. FIG. 15B is a top view of cardiovascular implant device 500 including fin-type flow conditioners 510. FIG. 15C is a bottom view of cardiovascular implant device 500 including fin-type flow conditioners 510. FIG. 16A is a sectional view of heart H illustrating an example positioning at a valve site of cardiovascular implant device 500 including fin-type flow conditioners 510. FIG. 16B is a sectional view of heart H illustrating an example positioning at a non-valve site of cardiovascular implant device 500 including fin-type flow conditioners 510.

As illustrated in FIGS. 15A-15C, cardiovascular implant device 500 includes fin-type flow conditioners 510, frame 512, cover 514, valvular body 516, inflow end 518, and outflow end 520. Frame 512 includes struts 522, inner diameter 524, inner surface 525, outer diameter 526, and outer surface 527, and defines openings 528, central flow path 529, and flow axis 530. Valvular body includes leaflets 531. FIG. 16A also shows device 500, heart H, right atrium RA, right ventricle RV, left atrium LA, left ventricle LV, superior vena cava SVC, inferior vena cava IVC, tricuspid valve TV, mitral valve MV, aortic valve AV, and aorta AT. FIG. 16B also shows device 500, heart H, vasculature V, right atrium RA, right ventricle RV, left atrium LA, left ventricle LV, superior vena cava SVC, inferior vena cava IVC, tricuspid valve TV, pulmonary valve PV, pulmonary artery PA, pulmonary veins PVS, mitral valve MV, aortic valve AV, aorta AT, and coronary sinus CS.

Cardiovascular implant device 500 is an implantable device for use in a cardiovascular system. Cardiovascular implant device 500 is configured to be implanted in blood vessels or chambers of heart H. In the illustrated example, cardiovascular implant device 500 is a valve device, such as a prosthetic valve device. In some examples, device 500 is deployed into a valve seat of a previously implanted prestent or docking station device (e.g., devices 100, 200, 300, and 400). Cardiovascular implant device 500 can be delivered into the cardiovascular system via a catheter (i.e., transcatheter delivery) or can be surgically placed using transcatheter or surgical procedures known in the art. In some examples, device 500 can be delivered and/or implanted using the same catheter or surgical procedure that is used for a prestent device (e.g., devices 100, 200, 300, and 400). In other examples, device 500 can be delivered and/or implanted by a separate catheter or in a separate surgical procedure. Device 500 can be located in any vessel or chamber of heart H at a site in heart H where there is not naturally a valve (a “non-valve” site) or at a site where there is a natural valve (e.g., aortic valve AV, mitral valve MV, pulmonary valve PV, etc.). For example, FIG. 16A shows an example positioning of device 500 at aortic valve AV (a valve site). In contrast, FIG. 16B shows an example positioning of device 500 in inferior vena cava IVC (a non-valve site).

Frame 512 forms a main body of device 500. Frame 512 can be expandable. Frame 512 can have a wide variety of different shapes and sizes. As shown in FIGS. 15A-15C, e.g., frame 512 is an annular or cylindrical mesh or lattice. Frame 512 has inner diameter 524 and outer diameter 526. Each of inner diameter 524 and outer diameter 526 can vary along a length of frame 512. Inner diameter 524 is a diameter of radially inner surface 525 of frame 512. Outer diameter 526 is a diameter of radially outer surface 527 of frame 512. Frame 512 can have any suitable length. For example, frame 512 may be approximately as long as valvular body 516. In other examples, frame 512 can be longer than valvular body 516. Frame 512 can press against or into tissue walls at the implant site or contour around anatomical structures of the cardiovascular system to set and maintain the position of device 500.

Frame 512 can be formed in a variety of ways, e.g., connecting individual wires together to form a mesh or lattice, braiding, cutting from a sheet and then rolling or otherwise forming into the shape of frame 512, molding, cutting from a cylindrical tube (e.g., cutting from a nitinol tube), other ways, or a combination of these. Frame 512 can be made from a highly flexible metal, metal alloy, or polymer. Examples of metals and metal alloys that can be used include, but are not limited to, nitinol and other shape-memory alloys, elgiloy, and stainless steel, but other metals and highly resilient or compliant non-metal materials can be used to make frame 512. All or a portion of frame 512 can be monolithically formed of any of these materials. These materials can allow frame 512 to be compressed to a small size, and then-when the compression force is released-frame 512 can self-expand back to its pre-compressed shape. Frame 512 can be expanded back to its pre-compressed shape due to the material properties of the material frame 112 is made out of and/or frame 512 can be expanded by inflation or expansion of a device positioned inside the frame. For example, frame 512 can be compressed such that frame 512 can fit into a delivery catheter. Frame 512 can also be made of other materials and can be expandable and collapsible in different ways, e.g., mechanically-expandable, balloon-expandable, self-expandable, or a combination of these.

Frame 512 extends between inflow end 518 and outflow end 520 of cardiovascular implant device 500. Inflow end 518 can be an end of device 500 that is relatively upstream of outflow end 520 with respect to a flow of blood along flow axis 530, as represented by arrow A in FIG. 15A, when device 500 is implanted in a blood vessel or chamber of heart H. Accordingly, outflow end 520 is an end of device 500 that is relatively downstream of inflow end 518 with respect to a flow of blood along flow axis 530, as represented by arrow A in FIG. 15A, when device 500 is implanted in a blood vessel or chamber of heart H. In the example shown in FIG. 16A, outflow end 520 is positioned within aorta AT and inflow end 518 is positioned upstream at the site of aortic valve AV (facing left ventricle LV). In the example shown in FIG. 16B, outflow end 520 is positioned near where inferior vena cava IVC opens into right atrium RA and inflow end 518 is positioned upstream within inferior vena cava IVC. Although inflow end 518 is defined as being relatively upstream of outflow end 520, it should be understood that other actual positions of inflow end 518 or outflow end 520 are possible depending on the location where device 500 is implanted.

Frame 512 is formed of a plurality of struts 522. Struts 522 make up the lattice or mesh of frame 512 and define openings (or cells) 528 therein. Struts 522 can be integrally formed. In some examples, all or a portion of struts 522 are monolithically formed from the same material. Openings 528 extend through frame 512 from inner surface 525 to outer surface 527. Each of openings 528 is bounded on one or more sides by ones of struts 522. Openings 528 can have any suitable shape or size, which can in turn be based on an overall shape or size of frame 512. In the example shown in FIG. 15A, openings 528 are a combination of hexagonal and diamond shaped and arranged in circumferential rows around frame 512. In other examples, openings 528 can have any other regular or irregular polygonal or non-polygonal shape and pattern. In some examples, ones of openings 528 can have different shapes or sizes throughout frame 512. For example, FIG. 15A shows a first row of openings 528 (adjacent outflow end 520) with openings 528 that are hexagonal shaped and the remaining rows of openings 528 with openings 528 that are diamond shaped and smaller. In some examples, ones of openings 528 can be connected by gaps to adjacent ones of openings 528.

Central flow path 529 is an open channel through a central portion of annular frame 512. Central flow path 529 is defined by inner surface 525 of frame 512. Central flow path 529 extends from inflow end 518 to outflow end 520 such that device 500 is open at each end. Accordingly, blood flowing through and out of device 500 follows central flow path 529. More specifically, flow axis 530 is a longitudinal axis through device 500 along which blood flows as it passes or is directed through device 500 (e.g., in the direction indicated by arrow A in FIG. 15A). In the example illustrated in FIG. 16A where device 500 is implanted at the site of aortic valve AV, device 500 can be closed (i.e., leaflets 531 of valvular body 516 can close) when heart H is in a diastolic phase. Blood flows from left atrium LA through mitral valve MV into left ventricle LV during the diastolic phase. During a systolic phase of heart H, device 500 can open. Blood flows from left ventricle LV through device 500 along flow axis 530 into aorta AT. In the example illustrated in FIG. 16B where device 500 is implanted in inferior vena cava IVC, device 500 can open when heart H is in a diastolic phase. Blood flows from inferior vena cava IVC and superior vena cava SVC into right atrium RA. The blood that flows from inferior vena cava IVC flows through device 500 along flow axis 530. During the diastolic phase, blood in right atrium RA flows through tricuspid valve TV and into right ventricle RV. During a systolic phase of heart H, device 500 can close. Blood is prevented from flowing (i.e., backflowing) from right atrium RA into inferior vena cava IVC by the closed device 500. A closed device 500 prevents any blood that regurgitates through the through tricuspid valve TV during the systolic phase from being forced into inferior vena cava IVC.

Cover 514 is a covering for one or more portions of frame 512. Cover 514 can be a fabric material, a polymer material, or other material. For example, cover 514 can be a material that promotes tissue ingrowth where device 500 contacts adjacent tissue walls of a vessel or chamber of heart H. Cover 514 can also form a seal to limit or prevent blood flow through portions of frame 512 that are covered by cover 514. Cover 514 can be attached to frame 512 by any suitable attachment means, such as by stitching, gluing, tying, etc. Cover 514 can be shaped and positioned in a variety of ways. In the example shown in FIG. 15A, cover 514 is adjacent to inflow end 518. In some examples, cover 514 is near or adjacent an attachment region for valvular body 516. In other examples, cover 514 can be adjacent to outflow end 520 or at any location or locations between inflow end 518 and outflow end 520. In yet other examples, device 500 does not include cover 514.

Valvular body 516 is mounted within annular frame 512. More specifically, valvular body 516 is connected to inner surface 525 of frame 512. Valvular body 516 includes one or more leaflets 531. In the example shown in FIGS. 15A-15C, there are three leaflets 531 (i.e., a tricuspid arrangement). In other examples, valvular body 516 can include more or fewer leaflets 531. The plurality of leaflets 531 are flexible and collapsible within frame 512 to regulate the flow of blood through device 500.

Fin-type flow conditioners 510 can generally include the same structure and function as fin-type flow conditioners 110 shown in FIGS. 3A-4, fin-type flow conditioner 140 shown in FIGS. 5A-5B, fin-type flow conditioner 140′ shown in FIGS. 6A-8, and fin-type flow conditioners 185A-185E shown in FIGS. 9A-9E. One or more flow conditioners 510 can be positioned in any suitable arrangement with respect to frame 512 of device 500. In some examples, flow conditioners 510 can be arranged about a circumference of inner surface 525 of frame 512 that is defined by inner diameter 524. In some examples, flow conditioners 510 can be located adjacent inflow end 518 (as shown in FIG. 15C) and/or outflow end 520 (as shown in FIG. 15B). In some examples, flow conditioners 510 are connected to ones of struts 522 that form a first row of openings 528 that is adjacent to outflow end 520. Device 500 can include any number of flow conditioners 510 in any one or more of the foregoing locations. Locations of flow conditioners 510 can be configured to prevent flow conditioners 510 from interfering with other parts of device 500 or adjacent tissue walls. For example, flow conditioners 510 can be positioned (or sized or shaped) to avoid interaction with valvular body 516 and leaflets 531. In other examples, components of device 500 (e.g., cover 514) can be designed to fit around flow conditioners 510 or to permit flow conditioners 510 to pass through. The locations of flow conditioners 510 can further be configured to allow flow conditioners 510 to collapse and expand with expandable frame 512 (e.g., to fit within a delivery catheter). The locations of flow conditioners 510 can further be configured to prevent flow conditioners 510 from occluding a vessel of chamber of heart H in which device 500 is implanted. The locations of flow conditioners 510 can further be configured to modify a hemodynamic characteristic (e.g., helicity, vorticity, velocity, turbulence, flow direction, etc.) of blood flowing through or out of device 500 in a particular manner.

Once device 500 is implanted in cardiovascular system (e.g., in aorta AT at aortic valve AV as shown in FIG. 16A or in inferior vena cava IVC as shown in FIG. 16B), circulating blood is delivered through device 500. As blood flows into, through, and out of device 500 along flow axis 530, flow conditioners 510 interact with the blood flow to modify or affect a hemodynamic characteristic (e.g., helicity, vorticity, velocity, turbulence, flow direction, etc.) of the flow. Flow conditioners 510 can interact with blood flowing through or out of device 500 by adding flow resistance and/or changing the direction of the blood flow to prevent reversal of blood flow. For example, flow conditioners 510 may increase or decrease vorticity or helicity of the flow. In some examples, flow conditioners 510 may cause the flow to be smoother (decrease the turbulence). In other examples, flow conditioners 510 can increase turbulence in the flow. In some examples, flow conditioners 510 can change a flow direction of the flow. In some examples, flow conditioners 510 can align the flow with a natural vortical flow pattern of blood through a vessel or chamber of heart H, such as the left-sided flow vortex in left atrium LA or the right-sided flow vortex in right atrium RA. In other examples, flow conditioners 510 can align the flow with a natural helical flow pattern of blood through a vessel or chamber of heart H, such as helical flow in coronary sinus CS. Flow conditioners 510 that are located circumferentially at inflow end 518 and/or outflow end 520 of device 500 can create helical flow patterns near an adjacent vessel or chamber wall to force blood to flow back towards the center of the vessel or chamber in a same helical direction, thereby producing forward movement of the blood. More generally, flow conditioners 510 adjacent to inflow end 518 can modify a hemodynamic characteristic of blood flowing through frame 512, and flow conditioners 510 adjacent to outflow end 520 can modify a hemodynamic characteristic of blood flowing out of frame 512.

In addition to the benefits described above with respect to devices 100 and 300, incorporating flow conditioners 510 directly on a valve device (e.g., device 500) provides an alternative or additional option to incorporating flow conditioners on a prestent or docking station device (e.g., devices 100 and 300). Flow conditioners 510 incorporated on device 500 can produce hemodynamic effects to minimize disruption to or enhance the natural flow patterns in scenarios where a prosthetic valve is implanted without a prestent or docking station.

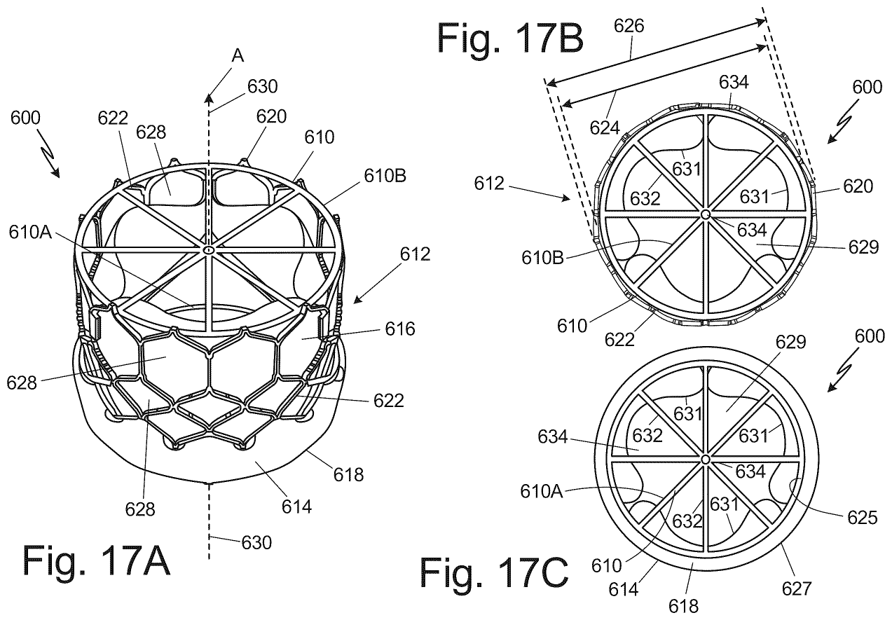

### DEVICE 600 (FIGS. 17A-17C)

FIGS. 17A-17C will be described together. FIG. 17A is a perspective view of cardiovascular implant device 600 including plate-type flow conditioners 610. FIG. 17B is a top view of cardiovascular implant device 600 including plate-type flow conditioners 610. FIG. 17C is a bottom view of cardiovascular implant device 600 including plate-type flow conditioners 610. As illustrated in FIGS. 17A-17C, cardiovascular implant device 600 includes plate-type flow conditioners 610, including flow conditioner 610A and 610B (which will be referred to collectively herein by the shared reference number), frame 612, cover 614, valvular body 616, inflow end 618, and outflow end 620. Frame 612 includes struts 622, inner diameter 624, inner surface 625, outer diameter 626, and outer surface 627, and defines openings 628, central flow path 629, and flow axis 630. Valvular body includes leaflets 631. Flow conditioners 610 include walls 632 and define flow passages 634 therein.

Cardiovascular implant device 600 includes a similar structure and function to cardiovascular implant device 500 described above, except device 600 includes plate-type flow conditioners 610 instead of fin-type flow conditioners (e.g., flow conditioners 510). Further, plate-type flow conditioners 610 can generally include the same structure and function as plate-type flow conditioners 210 shown in FIGS. 10A-10C and plate-type flow conditioners 285A-285E (FIGS. 11A-11E). One or more flow conditioners 610 can be positioned in any suitable arrangement with respect to frame 612 of device 600. In the example shown in FIG. 17A, device 600 includes two flow conditioners: flow conditioner 610A and flow conditioner 610B. Other examples can include any number of flow conditioners 610. Flow conditioners 610 are positioned to span across a portion of central flow path 629 (defined by frame 612) such that flow conditioners 610 intersect flow axis 630 through frame 612. In some examples, flow conditioners 610 can be located adjacent inflow end 618 (flow conditioner 610A as shown in FIG. 17C) and/or outflow end 620 (flow conditioner 610B as shown in FIG. 17B). In some examples, flow conditioners 610 are connected to ones of struts 622 that form a first row of openings 628 that is adjacent to outflow end 620. Device 600 can include any number of flow conditioners 610 in any one or more of the foregoing locations. Locations of flow conditioners 610 can be configured to prevent flow conditioners 610 from interfering (or contacting) other parts of device 600 or adjacent tissue walls. For example, flow conditioners 610 can be positioned (or sized or shaped) to avoid interaction with valvular body 616 and leaflets 631. In other examples, components of device 600 (e.g., cover 614) can be designed to fit around flow conditioners 610 or to permit flow conditioners 610 to pass through. The locations of flow conditioners 610 can further be configured to allow flow conditioners 610 to collapse and expand with expandable frame 612 (e.g., to fit within a delivery catheter). The locations of flow conditioners 610 can further be configured to prevent flow conditioners 610 from occluding a vessel of chamber of heart H in which device 600 is implanted. The locations of flow conditioners 610 can further be configured to modify a hemodynamic characteristic (e.g., helicity, vorticity, velocity, turbulence, flow direction, etc.) of blood flowing through or out of device 600 in a particular manner.

Once device 600 is implanted in the cardiovascular system, circulating blood is delivered through device 600. As blood flows into, through, and out of device 600 along flow axis 630, the blood flows through flow passages 634 of flow conditioners 610. Flow conditioners 610 interact with the blood flow to modify or affect a hemodynamic characteristic (e.g., helicity, vorticity, velocity, turbulence, flow direction, etc.) of the flow. Flow conditioners 610 can interact with blood flowing through or out of device 600 by adding flow resistance and/or changing the direction of the blood flow to prevent reversal of blood flow. For example, flow conditioners 610 may increase or decrease vorticity or helicity of the flow. In some examples, flow conditioners 610 may cause the flow to be smoother (decrease the turbulence). In other examples, flow conditioners 610 can increase turbulence in the flow. In some examples, flow conditioners 610 can change a flow direction of the flow. In some examples, flow conditioners 610 can align the flow with a natural vortical flow pattern of blood through a vessel or chamber of heart H, such as the left-sided flow vortex in left atrium LA or the right-sided flow vortex in right atrium RA. In other examples, flow conditioners 610 can align the flow with a natural helical flow pattern of blood through a vessel or chamber of heart H, such as helical flow in coronary sinus CS. Flow conditioners 610 that are located at inflow end 618 and/or outflow end 620 of device 600 can create helical flow patterns near an adjacent vessel or chamber wall to force blood to flow back towards the center of the vessel or chamber in a same helical direction, thereby producing forward movement of the blood. More generally, flow conditioners 610 adjacent to inflow end 618 can modify a hemodynamic characteristic of blood flowing through frame 612, and flow conditioners 610 adjacent to outflow end 620 can modify a hemodynamic characteristic of blood flowing out of frame 612.

In addition to the benefits described above with respect to devices 200 and 400, incorporating flow conditioners 610 directly on a valve device (e.g., device 600) provides an alternative or additional option to incorporating flow conditioners on a prestent or docking station device (e.g., devices 200 and 400). Flow conditioners 610 incorporated on device 600 can produce hemodynamic effects to minimize disruption to or enhance the natural flow patterns in scenarios where a prosthetic valve is implanted without a prestent or docking station.

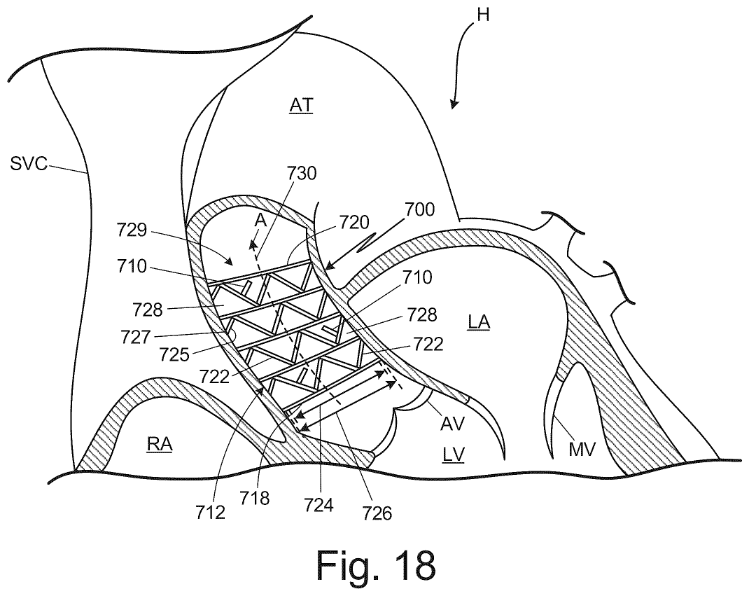

### DEVICE 700 (FIG. 18)

FIG. 18 is a sectional view of heart H illustrating an example positioning of cardiovascular implant device 700 including fin-type flow conditioners 710. As illustrated in FIGS. 18, cardiovascular implant device 700 includes fin-type flow conditioners 710, frame 712, inflow end 718, and outflow end 720. Frame 712 includes struts 722, inner diameter 724, inner surface 725, outer diameter 726, and outer surface 727, and defines openings 728, central flow path 729, and flow axis 730. FIG. 18 also shows device 700, heart H, right atrium RA, left atrium LA, left ventricle LV, superior vena cava SVC, mitral valve MV, aortic valve AV, and aorta AT.

Cardiovascular implant device 700 is an implantable device for use in a cardiovascular system. Cardiovascular implant device 700 is configured to be implanted in blood vessels or chambers of heart H. In the illustrated example, cardiovascular implant device 700 is a stent device. Cardiovascular implant device 700 can be delivered into the cardiovascular system via a catheter (i.e., transcatheter delivery) or can be surgically placed using transcatheter or surgical procedures known in the art. In some examples, device 700 can be delivered and/or implanted using the same catheter or surgical procedure that is used for an adjacent (or nearby) prestent device (e.g., devices 100, 200, 300, and 400) or a valve device (e.g., devices 500 and 600). In other examples, device 700 can be delivered and/or implanted by a separate catheter or in a separate surgical procedure. Device 700 can be located in any vessel or chamber of heart H. In some examples, device 700 is located at a site in heart H where there is not naturally a valve (a “non-valve” site). In other examples, device 700 is located near a site where there is a natural valve (e.g., near aortic valve AV, mitral valve MV, pulmonary valve PV, etc.). For example, FIG. 18 shows an example positioning of device 700 within aorta AT.

Frame 712 forms a main body of device 700. Frame 712 can be expandable. Frame 712 can have a wide variety of different shapes and sizes. As shown in FIG. 18, e.g., frame 712 is an annular or cylindrical mesh or lattice. Frame 712 has inner diameter 724 and outer diameter 726. Each of inner diameter 724 and outer diameter 726 can vary along a length of frame 712. Inner diameter 724 is a diameter of radially inner surface 725 of frame 712. Outer diameter 726 is a diameter of radially outer surface 727 of frame 712. Frame 712 can have any suitable length. Frame 712 can press against or into tissue walls at the implant site or contour (or extend) around anatomical structures of the cardiovascular system to set and maintain the position of device 700.

Frame 712 can be formed in a variety of ways, e.g., connecting individual wires together to form a mesh or lattice, braiding, cutting from a sheet and then rolling or otherwise forming into the shape of frame 712, molding, cutting from a cylindrical tube (e.g., cutting from a nitinol tube), other ways, or a combination of these. Frame 712 can be made from a highly flexible metal, metal alloy, or polymer. Examples of metals and metal alloys that can be used include, but are not limited to, nitinol and other shape-memory alloys, elgiloy, and stainless steel, but other metals and highly resilient or compliant non-metal materials can be used to make frame 712. All or a portion of frame 712 can be monolithically formed of any of these materials. These materials can allow frame 712 to be compressed to a small size, and then-when the compression force is released-frame 712 can self-expand back to its pre-compressed shape. Frame 712 can expand back to its pre-compressed shape due to the material properties of the material frame 712 is made out of and/or frame 712 can be expanded by inflation or expansion of a device positioned inside frame 712. For example, frame 712 can be compressed such that frame 712 can fit into a delivery catheter. Frame 712 can also be made of other materials and can be expandable and collapsible in different ways, e.g., mechanically-expandable, balloon-expandable, self-expandable, or a combination of these.

Frame 712 extends between inflow end 718 and outflow end 720 of cardiovascular implant device 700. Inflow end 718 can be an end of device 700 that is relatively upstream of outflow end 720 with respect to a flow of blood along flow axis 730, as represented by arrow A in FIG. 18, when device 700 is implanted in a blood vessel or chamber of heart H. Accordingly, outflow end 720 is an end of device 700 that is relatively downstream of inflow end 718 with respect to a flow of blood along flow axis 730, as represented by arrow A in FIG. 18, when device 700 is implanted in a blood vessel or chamber of heart H. In the example shown in FIG. 18, outflow end 720 is positioned within aorta AT and inflow end 718 is positioned upstream nearer to the site of aortic valve AV. Although inflow end 718 is defined as being relatively upstream of outflow end 720, it should be understood that other actual positions of inflow end 718 or outflow end 720 are possible depending on the location where device 700 is implanted.

Frame 712 is formed of a plurality of struts 722. Struts 722 make up the lattice or mesh of frame 712 and define openings (or cells) 728 therein. Struts 722 can be integrally formed. In some examples, all or a portion of struts 722 are monolithically formed from the same material. Openings 728 extend through frame 712 from inner surface 725 to outer surface 727. Each of openings 728 is bounded on one or more sides by ones of struts 722. Openings 728 can have any suitable shape or size, which can in turn be based on an overall shape or size of frame 712. In the example shown in FIG. 18, openings 728 are triangular and arranged in circumferential rows around frame 712. In other examples, openings 728 can have any other regular or irregular polygonal or non-polygonal shape and pattern. In some examples, ones of openings 728 can have different shapes or sizes throughout frame 712. For example, FIG. 18 shows varying dimensions of openings 728 throughout frame 712. In some examples, ones of openings 728 can be connected by gaps to adjacent ones of openings 728.

Central flow path 729 is an open channel through a central portion of annular frame 712. Central flow path 729 is defined by inner surface 725 of frame 712. Central flow path 729 extends from inflow end 718 to outflow end 720 such that device 700 is open at each end. Accordingly, blood flowing through and out of device 700 follows central flow path 729. More specifically, flow axis 730 is a longitudinal axis through device 700 along which blood flows as it passes or is directed through device 700 (e.g., in the direction indicated by arrow A in FIG. 18). In the example illustrated in FIG. 18 where device 700 is implanted in aorta AT, aortic valve AV (or a prosthetic valve device, such as device 500) opens during a systolic phase of heart H. Blood flows from left ventricle LV through aortic valve AV into aorta AT. Within aorta AT, blood flows through device 700 along flow axis 730.

Although not shown in FIG. 18, device 700 can also include a cover, which can generally include the same structure and function as covers 114 and 514 described above.

Fin-type flow conditioners 710 can generally include the same structure and function as fin-type flow conditioners 110 shown in FIGS. 3A-4, fin-type flow conditioner 140 shown in FIGS. 5A-5B, fin-type flow conditioner 140′ shown in FIGS. 6A-8, and fin-type flow conditioners 185A-185E shown in FIGS. 9A-9E. One or more flow conditioners 710 can be positioned in any suitable arrangement with respect to frame 712 of device 700. In some examples, flow conditioners 710 can be arranged about a circumference of inner surface 725 of frame 712 that is defined by inner diameter 724. In some examples, flow conditioners 710 can be located adjacent inflow end 718 and/or outflow end 720. In some examples, flow conditioners 710 are connected to ones of struts 722 that form a first row of openings 728 that is adjacent to outflow end 720. Device 700 can include any number of flow conditioners 710 in any one or more of the foregoing locations. Locations of flow conditioners 710 can be configured to prevent flow conditioners 710 from interfering with other parts of device 700 or adjacent tissue walls. In other examples, components of device 700 can be designed to fit around flow conditioners 710 or to permit flow conditioners 710 to pass through. The locations of flow conditioners 710 can further be configured to allow flow conditioners 710 to collapse and expand with expandable frame 712 (e.g., to fit within a delivery catheter). The locations of flow conditioners 710 can further be configured to prevent flow conditioners 710 from occluding a vessel of chamber of heart H in which device 700 is implanted. The locations of flow conditioners 710 can further be configured to modify a hemodynamic characteristic (e.g., helicity, vorticity, velocity, turbulence, flow direction, etc.) of blood flowing through or out of device 700 in a particular manner.

Once device 700 is implanted in the cardiovascular system (e.g., in aorta AT as shown in FIG. 18), circulating blood passes through device 700. As blood flows into, through, and out of device 700 along flow axis 730, flow conditioners 710 interact with the blood flow to modify or affect a hemodynamic characteristic (e.g., helicity, vorticity, velocity, turbulence, flow direction, etc.) of the flow. Flow conditioners 710 can interact with blood flowing through or out of device 700 by adding flow resistance and/or changing the direction of the blood flow to prevent reversal of blood flow. For example, flow conditioners 710 may increase or decrease vorticity or helicity of the flow. In some examples, flow conditioners 710 may cause the flow to be smoother (decrease the turbulence). In other examples, flow conditioners 710 can increase turbulence in the flow. In some examples, flow conditioners 710 can change a flow direction of the flow. In some examples, flow conditioners 710 can align the flow with a natural vortical flow pattern of blood through a vessel or chamber of heart H, such as the left-sided flow vortex in left atrium LA or the right-sided flow vortex in right atrium RA. In other examples, flow conditioners 710 can align the flow with a natural helical flow pattern of blood through a vessel or chamber of heart H, such as helical flow in coronary sinus CS. Flow conditioners 710 that are located circumferentially at inflow end 718 and/or outflow end 720 of device 700 can create helical flow patterns near an adjacent vessel or chamber wall to force blood to flow back towards the center of the vessel or chamber in a same helical direction, thereby producing forward movement of the blood. More generally, flow conditioners 710 adjacent to inflow end 718 can modify a hemodynamic characteristic of blood flowing through frame 712, and flow conditioners 710 adjacent to outflow end 720 can modify a hemodynamic characteristic of blood flowing out of frame 712.

In addition to the benefits described above with respect to devices 100 and 300, incorporating flow conditioners 710 directly on a stent device (e.g., device 700) can produce hemodynamic effects to minimize disruption to or enhance the natural flow patterns at any sites where a stent may be implanted. To some extent, this may be a greater variety of locations throughout the cardiovascular system (e.g., any vessel or chamber of heart H), compared to valve devices or prestent/docking station devices which may have more limited applications.

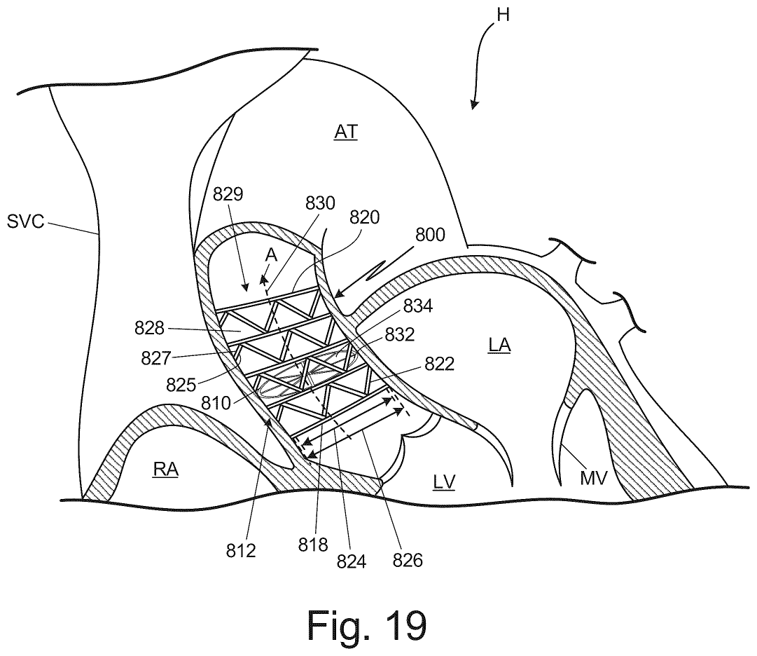

### DEVICE 800 (FIG. 19)

FIG. 19 is a sectional view of heart H illustrating an example positioning of cardiovascular implant device 800 including plate-type flow conditioner 810. As illustrated in FIG. 19, cardiovascular implant device 800 includes plate-type flow conditioner 810, frame 812, inflow end 818, and outflow end 820. Frame 812 includes struts 822, inner diameter 824, inner surface 825, outer diameter 826, and outer surface 827, and defines openings 828, central flow path 829, and flow axis 830. Flow conditioner 810 includes walls 832 and define flow passages 834 therein. FIG. 19 also shows device 800, heart H, right atrium RA, left atrium LA, left ventricle LV, superior vena cava SVC, mitral valve MV, aortic valve AV, and aorta AT.

Cardiovascular implant device 800 includes a similar structure and function to cardiovascular implant device 700 described above, except cardiovascular implant device 800 includes plate-type flow conditioner 810 instead of fin-type flow conditioners (e.g., flow conditioners 710). In the example shown in FIG. 19, device 800 includes one flow conditioner 810. Other examples can include any number of flow conditioners 810. Further, plate-type flow conditioner 810 can generally include the same structure and function as plate-type flow conditioners 210 shown in FIGS. 10A-10C and plate-type flow conditioners 285A-285E shown in FIGS. 11A-11E. One or more flow conditioners 810 can be positioned in any suitable arrangement with respect to frame 812 of device 800. Flow conditioners 810 are positioned to span across a portion of central flow path 829 (defined by frame 812) such that flow conditioners 810 intersect flow axis 830 through frame 812. In some examples, flow conditioners 810 can be located adjacent inflow end 818 and/or outflow end 620. In some examples, flow conditioners 810 are connected to ones of struts 822 that form a first row of openings 828 that is adjacent to outflow end 820. Device 800 can include any number of flow conditioners 810 in any one or more of the foregoing locations. Locations of flow conditioners 810 can be configured to prevent flow conditioners 810 from interfering (or contacting) other parts of device 800 or adjacent tissue walls. In other examples, components of device 800 can be designed to fit around flow conditioners 810 or to permit flow conditioners 810 to pass through. The locations of flow conditioners 810 can further be configured to allow flow conditioners 810 to collapse and expand with expandable frame 812 (e.g., to fit within a delivery catheter). The locations of flow conditioners 810 can further be configured to prevent flow conditioners 810 from occluding a vessel of chamber of heart H in which device 800 is implanted. The locations of flow conditioners 810 can further be configured to modify a hemodynamic characteristic (e.g., helicity, vorticity, velocity, turbulence, flow direction, etc.) of blood flowing through or out of device 800 in a particular manner.

Once device 800 is implanted in the cardiovascular system (e.g., in aorta AT as shown in FIG. 19), circulating blood passes through device 800. As blood flows into, through, and out of device 800 along flow axis 830, the blood flows through flow passages 834 of flow conditioners 810. Flow conditioner 810 interacts with the blood flow to modify or affect a hemodynamic characteristic (e.g., helicity, vorticity, velocity, turbulence, flow direction, etc.) of the flow. Flow conditioner 810 can interact with blood flowing through or out of device 800 by adding flow resistance and/or changing the direction of the blood flow to prevent reversal of blood flow. For example, flow conditioner 810 may increase or decrease vorticity or helicity of the flow. In some examples, flow conditioner 810 may cause the flow to be smoother (decrease the turbulence). In other examples, flow conditioner 810 can increase turbulence in the flow. In some examples, flow conditioner 810 can change a flow direction of the flow. In some examples, flow conditioner 810 can align the flow with a natural vortical flow pattern of blood through a vessel or chamber of heart H, such as the left-sided flow vortex in left atrium LA or the right-sided flow vortex in right atrium RA. In other examples, flow conditioner 810 can align the flow with a natural helical flow pattern of blood through a vessel or chamber of heart H, such as helical flow in coronary sinus CS. Flow conditioners 810 that is located at inflow end 818 and/or outflow end 820 of device 800 can create helical flow patterns near an adjacent vessel or chamber wall to force blood to flow back towards the center of the vessel or chamber in a same helical direction, thereby producing forward movement of the blood. More generally, flow conditioner 810 adjacent to inflow end 818 can modify a hemodynamic characteristic of blood flowing through frame 812, and flow conditioner 810 adjacent to outflow end 820 can modify a hemodynamic characteristic of blood flowing out of frame 812.

In addition to the benefits described above with respect to devices 200 and 400, incorporating flow conditioner 810 directly on a stent device (e.g., device 800) can produce hemodynamic effects to minimize disruption to or enhance the natural flow patterns at any sites where a stent may be implanted. To some extent, this may be a greater variety of locations throughout the cardiovascular system (e.g., any vessel or chamber of heart H), compared to valve devices or prestent/docking station devices which may have more limited applications.

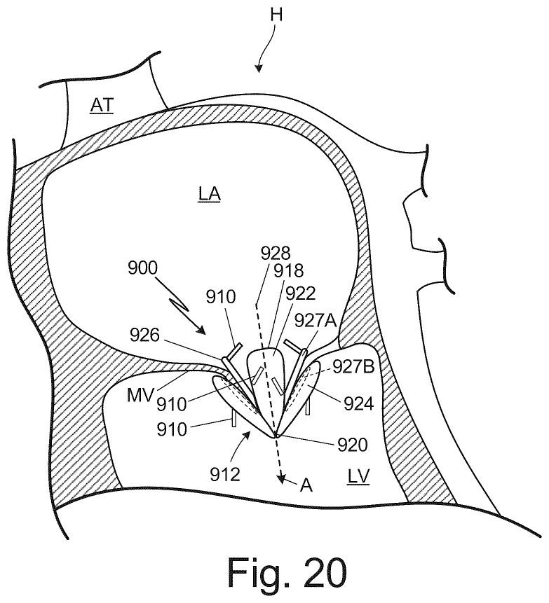

### DEVICE 900 (FIG. 20)

FIG. 20 is a sectional view of heart H illustrating an example positioning of cardiovascular implant device 900 including fin-type flow conditioners 910. As illustrated in FIG. 20, cardiovascular implant device 900 includes fin-type flow conditioners 910, body 912, inflow end 918, and outflow end 920. Body 912 includes central spacer 922, paddles 924, clasps 926 (including first arm 927A and second arm 927B), and central longitudinal axis 928. FIG. 20 also shows device 900, heart H, left atrium LA, left ventricle LV, mitral valve MV, and aorta AT.

Cardiovascular implant device 900 is an implantable device for use in a cardiovascular system. Cardiovascular implant device 900 is configured to be implanted in blood vessels or chambers of heart H. In the illustrated example, cardiovascular implant device 900 is an edge-to-edge valve repair device. Cardiovascular implant device 900 can be delivered into the cardiovascular system via a catheter (i.e., transcatheter delivery) or can be surgically placed using transcatheter or surgical procedures known in the art. In some examples, device 900 can be delivered and/or implanted using the same catheter or surgical procedure that is used for an adjacent (or nearby) stent device (e.g., devices 700 and 800). In other examples, device 900 can be delivered and/or implanted by a separate catheter or in a separate surgical procedure. Device 900 can be located in any vessel or chamber of heart H. In particular, device 900 is located near a site where there is a natural valve (e.g., near mitral valve MV, a tricuspid valve, an aortic valve, a pulmonary valve, etc.). For example, FIG. 20 shows an example positioning of device 900 attached to mitral valve MV. Other examples can include device 900 attached to other natural valves, such as a tricuspid valve, an aortic valve, a pulmonary valve, etc.

Body 912 forms a main body of device 900. Body 912 can be formed in a variety of ways and can be made from a highly flexible metal, metal alloy, or polymer. Examples of metals and metal alloys that can be used include, but are not limited to, nitinol and other shape-memory alloys, elgiloy, and stainless steel, but other metals and highly resilient or compliant non-metal materials can be used to make body 912. All or a portion of body 912 can be monolithically formed of any of these materials. Body 912, including central spacer 922, paddles 924, and clasps 926, can have an expanded and a closed or collapsed configuration. For example, body 912 can be sized or collapsed to fit into a delivery catheter in a closed configuration. Body 912 can, in some examples, be expanded during an implantation procedure to attach device 900 to a natural valve of heart H.

Body 912 includes central spacer 922. Central spacer 922 forms a central portion of device 900. Central spacer 922 can be generally elongated, cylindrical, or tapered in shape. Central spacer 922 is configured to extend through an opening between leaflets of a natural valve of heart H and maintain a separation between sets of paddles 924 and clasps 926 that bridges the opening between the leaflets. Central longitudinal axis 928 extends longitudinally through central spacer 922.

Clasps 926 are elongated projections from body 912 that extend radially outward from central spacer 922 and central longitudinal axis 928. Clasps 926 include a respective first arm 927A and second arm 927B arranged in a U-shape or V-shape. First arms 927A of clasps 926 are configured to contact or press against a first side of the leaflets of the natural valve of heart H. In the example shown in FIG. 20, first arms 927A contact a side of the leaflets of mitral valve MV that faces left atrium LA. Second arms 927B (shown in dashed lines behind a portion of paddle 924 in FIG. 20) of clasps 926 are configured to contact or press against a second side of the leaflets of the natural valve of heart H. In the example shown in FIG. 20, second arms 927B contact a side of the leaflets of mitral valve MV that faces left ventricle LV. Pairs of first arms 927A and second arms 927B function together to grip the leaflet of leaflets of the natural valve of heart H.

Paddles 924 are paddle shaped or elongated and relatively widened and flattened projections from body 912 that extend radially outward from central spacer 922 and central longitudinal axis 928. Paddles 924 are configured to contact or press against second arms 927B of clasps 926. In the example shown in FIG. 20, paddles 924 are positioned on a side of mitral valve MV that faces left ventricle LV such that paddles 924 are within left ventricle LV. Each of paddles 924 has a corresponding one of clasps 926 for securing device 900 to the leaflets and holding the leaflets together around device 900. As such, pairs of paddles 924 and corresponding clasps 926 can have complimentary shapes and/or sizes so each pair fits together to grip the leaflet or leaflets. Paddles 924 and clasps 926 can be independently or cooperatively actuated to have different angles with respect to central longitudinal axis 928. An angle of paddles 924 with respect to central longitudinal axis 928 can be adjusted based on the desired amount of contact (i.e., pressure) between paddles 924 and clasps 926 at second arms 927B.

Body 912 extends between inflow end 918 and outflow end 920 of cardiovascular implant device 900. Inflow end 918 can be an end of device 900 that is relatively upstream of outflow end 920 with respect to a flow of blood parallel to central longitudinal axis 928, as represented by arrow A in FIG. 20, when device 900 is implanted in a blood vessel or chamber of heart H. Accordingly, outflow end 920 is an end of device 900 that is relatively downstream of inflow end 918 with respect to a flow of blood parallel to central longitudinal axis 928, as represented by arrow A in FIG. 20, when device 900 is implanted in a blood vessel or chamber of heart H. In the example shown in FIG. 20, outflow end 920 is positioned within left ventricle LV and inflow end 918 is positioned upstream within left atrium LA, such that left atrium LA pumps blood through mitral valve MV, around device 900, and into left ventricle LV. Although inflow end 918 is defined as being relatively upstream of outflow end 920, it should be understood that other actual positions of inflow end 918 or outflow end 920 are possible depending on the location where device 900 is implanted.

Although not shown in FIG. 20, device 900 can also include a cover, which can generally include the same structure and function as covers 114 and 514 described above.

Fin-type flow conditioners 910 can generally include the same structure and function as fin-type flow conditioners 110 shown in FIGS. 3A-4, fin-type flow conditioner 140 shown in FIGS. 5A-5B, fin-type flow conditioner 140′ shown in FIGS. 6A-8, fin-type flow conditioners 185A-185E shown in FIGS. 9A-9E. One or more flow conditioners 910 can be positioned in any suitable arrangement with respect to body 912 of device 900. In some examples (e.g., as shown in FIG. 20), flow conditioners 910 can be connected to and arranged about central spacer 922, paddles 924, and/or clasps 926. In some examples, flow conditioners 910 can be connected circumferentially around central spacer 922 such that flow conditioners 910 extend radially outward with respect to central longitudinal axis 928. In some examples, flow conditioners 910 can be located adjacent inflow end 918 and/or outflow end 920. Device 900 can include any number of flow conditioners 910 in any one or more of the foregoing locations. Locations of flow conditioners 910 can be configured to prevent flow conditioners 910 from interfering with other parts of device 900 or adjacent tissue walls. In other examples, components of device 900 can be designed to fit around flow conditioners 910 or to permit flow conditioners 910 to pass through. The locations of flow conditioners 910 can further be configured to allow flow conditioners 910 to collapse and expand with body 912 (e.g., to fit within a delivery catheter). The locations of flow conditioners 910 can further be configured to prevent flow conditioners 910 from occluding a vessel of chamber of heart H in which device 900 is implanted. The locations of flow conditioners 910 can further be configured to modify a hemodynamic characteristic (e.g., helicity, vorticity, velocity, turbulence, flow direction, etc.) of blood flowing around device 900 in a particular manner.

Once device 900 is implanted in the cardiovascular system (e.g., attached to mitral valve MV as shown in FIG. 20), circulating blood passes around device 900. As blood flows around device 900 parallel to central longitudinal axis 928, flow conditioners 910 interact with the blood flow to modify or affect a hemodynamic characteristic (e.g., helicity, vorticity, velocity, turbulence, flow direction, etc.) of the flow. Flow conditioners 910 can interact with blood flowing around device 900 by adding flow resistance and/or changing the direction of the blood flow to prevent reversal of blood flow. For example, flow conditioners 910 may increase or decrease vorticity or helicity of the flow. In some examples, flow conditioners 910 may cause the flow to be smoother (decrease the turbulence). In other examples, flow conditioners 910 can increase turbulence in the flow. In some examples, flow conditioners 910 can change a flow direction of the flow. In some examples, flow conditioners 910 can align the flow with a natural vortical flow pattern of blood through a vessel or chamber of heart H, such as the left-sided flow vortex in left atrium LA or the right-sided flow vortex in right atrium RA. In other examples, flow conditioners 910 can align the flow with a natural helical flow pattern of blood through a vessel or chamber of heart H, such as helical flow in coronary sinus CS. Flow conditioners 910 that are located adjacent inflow end 918 and/or outflow end 920 of device 900 can create helical flow patterns near an adjacent vessel or chamber wall to force blood to flow back towards the center of the vessel or chamber in a same helical direction, thereby producing forward movement of the blood. More generally, flow conditioners 910 adjacent to inflow end 918 can modify a hemodynamic characteristic of blood flowing into a natural valve of heart H, and flow conditioners 910 adjacent to outflow end 920 can modify a hemodynamic characteristic of blood flowing out of a natural valve of heart H.

In addition to the benefits described above with respect to devices 100, 300, 500, and 700, incorporating flow conditioners 910 on an edge-to-edge valve repair device (e.g., device 900) can produce hemodynamic effects to minimize disruption to or enhance the natural flow patterns at natural valve sites prior to a valve replacement procedure. Device 900 including flow conditioners 910 can mitigate adverse changes on intraventricular flow dynamics that may be associated with increased left ventricular work.

Although depicted in FIGS. 3A-20 as separate examples, a cardiovascular implant device according to techniques of this disclosure can include any combination of the foregoing features, unless expressly limited. Valve devices (e.g., devices 500 and 600) and/or stents (e.g., devices 100, 200, 300, 400, 700, and 800) can be used alone or in tandem to restrict, enhance, or modulate flow in order to enhance or restore normal vortical and helical flow patterns. Moreover, dedicated flow conditioner devices (i.e., devices including flow conditioners described herein) can be used in patients without severe valvular regurgitation to delay or prevent progressive atrial and ventricular chamber remodeling. In other words, in patients with moderate regurgitation who are not yet candidates for edge-to-edge valve repair or valve replacement, flow conditioner devices described herein can mitigate the impact of the moderate regurgitation on cardiac remodeling. Any of the flow conditioner devices described herein can maintain kinetic energy of the cardiovascular blood flow, which in turn reduces the cardiac work needed and improves cardiac efficiency.

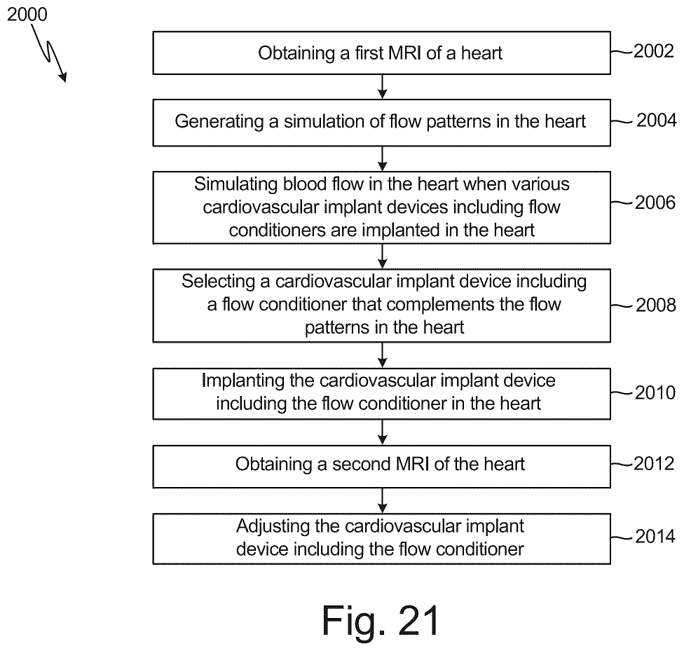

FIG. 21 is a flowchart showing method 2000 for selecting a cardiovascular implant device including a flow conditioner for implantation in the heart. Method 2000 includes steps 2002-2014. A cardiovascular implant device including a flow conditioner is selected according to method 2000 to minimize or eliminate disruption of or enhance flow patterns of blood flow in a heart of a patient.

Step 2002 includes obtaining a first MRI of a heart. The first MRI (magnetic resonance imaging) can visualize the flow patterns of blood flow in the heart of the patient. Specifically, the first MRI can visualize the flow patterns of blood flow in the vessels and/or chambers (right atrium, left atrium, right ventricle, and left ventricle) of the heart of the patient.

The first MRI can be a 4D MRI that visualizes the flow patterns of blood flow in the heart of a patient. Additionally, the 4D MRI can measure volumes in the chambers of the heart, sizes of the chambers of the heart, geometries of the chambers of the heart, compliances of the chambers of the heart, and/or blood pressures in the chambers of the heart. The 4D MRI can also track movement of the chambers of the heart and movement of the tricuspid valve (also known as tricuspid annular plan systolic excursion (TAPSE)).

Step 2004 includes generating a simulation of the flow patterns in the heart. The simulation of the flow patterns in the heart can be generated based on the first MRI. This allows the simulation to be patient specific. The simulation will simulate the flow patterns in the heart of the patient, such as in the vessels and/or chambers of the heart. The simulation can also simulate the volumes, sizes, geometries, compliances, and blood pressures of the chambers of the heart based on data from the first MRI. The simulation can be generated using any suitable software program.

Step 2006 includes simulating blood flow in the heart when various cardiovascular implant devices including flow conditioners are implanted in the heart. The simulated blood flow in the heart is modulated by the cardiovascular implant devices including the flow conditioners to simulate the impact of the cardiovascular implant devices and the flow conditioners on the flow patterns in the heart. The blood flow in the heart can be simulated when the heart includes cardiovascular implant devices including various types of flow conditioners (e.g., fin type and/or plate type) having varying forms (e.g., varying shapes, sizes, presence of flow microfeatures, etc.), varying physical dimensions, varying arrangements or positions with respect to the cardiovascular implant device, varying attachment or extension angles, and/or other possible variations described herein.

Step 2008 includes selecting the cardiovascular implant device including a flow conditioner (or multiple flow conditioners) that complements the flow patterns in the heart. The cardiovascular implant device including the flow conditioner is selected to minimize or eliminate disruption of or enhance flow patterns in the heart. Specifically, step 2008 can include selecting a design of the cardiovascular implant device including the flow conditioner that complements the flow patterns in the heart. More specifically, a type of the flow conditioner can be selected to complement the flow patterns in the heart; a form and/or physical dimension of the flow conditioner can be selected to complement the flow pattern in the heart; and an arrangement or position and/or angle of the flow conditioner can be selected to complement the flow pattern in the heart.

The right atrium of the heart has a right-sided flow vortex as a natural flow pattern in the heart. The design of the cardiovascular implant device including the flow conditioner can be selected to complement the right-sided flow vortex in the right atrium of the heart. The left atrium of the heart has a left-sided flow vortex as a natural flow pattern in the heart. The design of the cardiovascular implant device including the flow conditioner can be selected to complement the left-sided flow vortex in the left atrium of the heart. The coronary sinus and other vessels of the heart may have a helical flow pattern. The design of the cardiovascular implant device including the flow conditioner can be selected to complement the helical flow pattern in the coronary sinus or other vessels of the heart.

In an alternate example, step 2008 can include selecting a design of a cardiovascular implant device including a flow conditioner that enhances the flow pattern in the right atrium of the heart and/or reestablishes the natural flow pattern in the right atrium of the heart. Specifically, if the patient has lost the right-sided flow vortex of blood flow in the right atrium of the heart due to age, disease, or anatomical defects, the design of the cardiovascular implant device including the flow conditioner can be selected to reestablish the right-sided flow vortex of blood flow in the right atrium of the heart.

Step 2010 includes implanting the cardiovascular implant device including the flow conditioner in the heart. The cardiovascular implant device including the flow conditioner can be implanted using any suitable method.

Step 2012 includes obtaining a second MRI of the heart. The second MRI (magnetic resonance imaging) can visualize the flow patterns of blood flow in the heart of the patient after the cardiovascular implant device including the flow conditioner has been implanted. Specifically, the second MRI can visualize the flow patterns of blood flow in the vessels and/or chambers (right atrium, left atrium, right ventricle, and left ventricle) of the heart of the patient after the cardiovascular implant device including the flow conditioner has been implanted.

The second MRI can be a 4D MRI that visualizes the flow patterns of blood flow in the heart of a patient after the cardiovascular implant device including the flow conditioner has been implanted. Additionally, the 4D MRI can measure volumes in the chambers of the heart, sizes of the chambers of the heart, geometries of the chambers of the heart, compliances of the chambers of the heart, and/or blood pressures in the chambers of the heart. The 4D MRI can also track movement of the chambers of the heart and movement of the tricuspid valve (also known as tricuspid annular plan systolic excursion (TAPSE)).

The second MRI is obtained to confirm that the cardiovascular implant device including the flow conditioner complements (e.g., has minimal to no disruption of or enhances) the flow patterns in the heart. Further, the second MRI can be obtained to determine whether the cardiovascular implant device including the flow conditioner has enhanced and/or reestablished the natural flow patterns in the heart. Specifically, the second MRI can be obtained to determine whether the cardiovascular implant device including the flow conditioner has reestablished a right-sided flow vortex in a right atrium of the heart.

The second MRI can also confirm the overall health of the heart after the cardiovascular implant device including the flow conditioner has been implanted. Specifically, the volumes in the chambers of the heart, sizes of the chambers of the heart, geometries of the chambers of the heart, compliances of the chambers of the heart, and/or blood pressures in the chambers of the heart from the second MRI can be compared to the same readings from the first MRI to confirm overall health of the heart. In one example, the volumes, sizes, geometries, and/or compliances of the chambers of the heart can be analyzed to determine if the left side of the heart has experienced remodeling (shrinkage) due to the reduced blood pressure on the left side of the heart after the cardiovascular implant device including the flow conditioner has been implanted. Further, the volumes, sizes, geometries, and/or compliances of the chambers of the heart can be analyzed to determine if the right side of the heart is being overloaded due to the increased blood pressure in the right side of the heart.

Step 2014 includes adjusting the cardiovascular implant device including the flow conditioner. The cardiovascular implant device including the flow conditioner can be adjusted if the second MRI shows that the implantation of the cardiovascular implant device including the flow conditioner has not had the desired effect on the flow patterns in or overall health of the heart. In one example, a type of the flow conditioner can be changed, such as from one or more fin type, plate type, or deflector type flow conditioners or a combination of types to one or more of a different type of flow conditioner or a different combination of types. In another example, a form, physical dimension, and/or arrangement or position of the flow conditioner can be adjusted. In another example, the angle of the flow conditioner can be adjusted. For example, the flow conditioner can be connected to the cardiovascular implant device by an adjustable bias member that can be adjusted such that the flow conditioner has a different angle, or the flow conditioner can be electromechanically actuated to a different angle.

Method 2000 as described herewith can be used to aid in the selection and implantation of any suitable cardiovascular implant device including a flow conditioner. In one example, method 2000 can be used to aid in the selection and implantation of cardiovascular implant device 100 (shown in FIGS. 3A-9E), cardiovascular implant device 200 (shown in FIGS. 10A-11E), cardiovascular implant device 300 (shown in FIGS. 12-13), cardiovascular implant device 400 (shown in FIG. 14), cardiovascular implant device 500 (shown in FIGS. 15A-16B), cardiovascular implant device 600 (shown in FIGS. 17A-17C), cardiovascular implant device 700 (shown in FIG. 18), cardiovascular implant device 800 (shown in FIG. 19), or cardiovascular implant device 900 (shown in FIG. 20). Method 2000 can be used to select a design of cardiovascular implant device 100, 200, 300, 400, 500, 600, 700, 800, or 900 that will complement (e.g., minimize or eliminate disruptions or enhance) the flow patterns in the heart. In alternate examples, method 2000 can be used with any other design of a cardiovascular implant device including a flow conditioner.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.). That is, any of devices 100, 200, 300, 400, 500, 600, 700, 800, and 900 or components of devices 100, 200, 300, 400, 500, 600, 700, 800, and 900 can be sterilized before being delivered into the body.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Discussion of Possible Examples

The following are non-exclusive descriptions of possible examples of the present invention.

A cardiovascular implant device includes an expandable annular frame and a flow conditioner. The expandable annular frame is formed of a plurality of struts and is configured to conform to an interior shape of a blood vessel or a chamber of a heart when expanded inside the blood vessel or the chamber of the heart. The flow conditioner is connected to the plurality of struts of the expandable annular frame. The flow conditioner is positioned to modify a hemodynamic characteristic of a flow of blood through or out of the expandable annular frame.

The cardiovascular implant device of the preceding paragraph can optionally include, additionally and/or alternatively, any one or more of the following features, configurations, and/or additional components:

The flow conditioner can include one or more fins.

The one or more fins can be connected circumferentially about an interior of the expandable annular frame.

The one or more fins can be connected to ones of the plurality of struts that form a first row of openings in the expandable annular frame, the first row of openings being adjacent to an outflow end of the expandable annular frame.

The one or more fins can be angled radially inward from a circumference of the expandable annular frame.

The one or more fins can be connected adjacent to an inflow or outflow end of the expandable annular frame.

The one or more fins can be angled radially inward from the inflow or outflow end of the expandable annular frame.

The one or more fins can include a first fin connected to the expandable annular frame at an inflow end of the cardiovascular implant device to modify the hemodynamic characteristic of the flow of blood through the expandable annular frame and a second fin connected to the expandable annular frame at an outflow end of the cardiovascular implant device to modify the hemodynamic characteristic of the flow of blood out of the expandable annular frame.

Each fin of the one or more fins can be deflectable by the flow of blood through or out of the expandable annular frame.

Each fin of the one or more fins can be connected to the expandable annular frame by a spring.

The one or more fins can be airfoils, and the one or more fins can include flow microfeatures proximal to a leading edge of respective ones of the one or more fins.

The flow microfeatures can include at least one of a vortex generator, a leading edge notch, a leading edge dogtooth, a boundary layer fence, and a vortilon.

The flow conditioner and the expandable annular frame can form a monolithic structure.

The flow conditioner and a portion of the expandable annular frame to which the flow conditioner is connected can be formed of a shape-memory alloy.

The shape-memory alloy can be nitinol.

The flow conditioner can be electromechanically actuated.

An attachment angle of the flow conditioner as measured with respect to a longitudinal axis of a respective one of the plurality of struts to which the flow conditioner is connected can be controllable over a range of possible angles.

The flow conditioner can include one or more plates that span across a portion of the expandable annular frame such that the one or more plates intersect a flow axis through the expandable annular frame, the one or more plates each including a plurality of flow passages.

The one or more plates can include a first plate having a first plurality of flow passages and a second plate having a second plurality of flow passages, and the first plate can be connected at an inflow end of the cardiovascular implant device to modify the hemodynamic characteristic of the flow of blood through the expandable annular frame and the second plate can be connected to an outflow end of the cardiovascular implant device to modify the hemodynamic characteristic of the flow of blood out of the expandable annular frame.

The flow conditioner can be connected to the expandable annular frame at an inflow end of the cardiovascular implant device to modify the hemodynamic characteristic of the flow of blood through the expandable annular frame.

The flow conditioner can be connected to the expandable annular frame at an outflow end of the cardiovascular implant device to modify the hemodynamic characteristic of the flow of blood out of the expandable annular frame.

The flow conditioner can include a first flow conditioner feature connected to the expandable annular frame at an inflow end of the cardiovascular implant device and a second flow conditioner feature connected to the expandable annular frame at an outflow end of the cardiovascular implant device, and the first flow conditioner feature can be positioned to modify the hemodynamic characteristic of the flow of blood through the expandable annular frame and the second flow conditioner feature can be positioned to modify the hemodynamic characteristic of the flow of blood out of the expandable annular frame.

The flow conditioner can have a physical dimension that causes the flow conditioner to avoid interaction with an adjacent tissue wall.

The flow conditioner can include at least one of a fin and a plate, the plate including a plurality of flow passages.

The cardiovascular implant device can be sterilized.

The cardiovascular implant device can be a docking station configured to support an expandable transcatheter valve.

The cardiovascular implant device can be a stent.

The cardiovascular implant device can be configured to be implanted at a valve site.

The cardiovascular implant device can be configured to be implanted at a non-valve site.

A prosthetic valve device includes an annular frame formed of a plurality of struts, a valvular body mounted within the annular frame, and a flow conditioner. The valvular body includes a plurality of leaflets that regulate a flow of blood through the annular frame. The flow conditioner is connected to the plurality of struts of the annular frame. The flow conditioner is positioned to modify a hemodynamic characteristic of the flow of blood through or out of the annular frame.

The prosthetic valve device of the preceding paragraph can optionally include, additionally and/or alternatively, any one or more of the following features, configurations, and/or additional components:

The flow conditioner can include one or more fins.

The one or more fins can be connected circumferentially about an interior of the annular frame.

The one or more fins can be connected to ones of the plurality of struts that form a first row of openings in the annular frame, the first row of openings being adjacent to an outflow end of the annular frame.

The one or more fins can be angled radially inward from a circumference of the annular frame.

The one or more fins can be connected adjacent to an inflow or outflow end of the annular frame.

The one or more fins can be angled radially inward from the inflow or outflow end of the annular frame.

The one or more fins can include a first fin connected to the annular frame at an inflow end of the prosthetic valve device to modify the hemodynamic characteristic of the flow of blood through the annular frame and a second fin connected to the annular frame at an outflow end of the prosthetic valve device to modify the hemodynamic characteristic of the flow of blood out of the annular frame.

Each fin of the plurality of fins can be deflectable by the flow of blood through or out of the annular frame.

Each fin of the plurality of fins can be connected to the annular frame by a spring.

The one or more fins can be airfoils, and the one or more fins can include flow microfeatures proximal to a leading edge of respective ones of the one or more fins.

The flow microfeatures can include at least one of a vortex generator, a leading edge notch, a leading edge dogtooth, a boundary layer fence, and a vortilon.

The flow conditioner and the annular frame can form a monolithic structure.

The flow conditioner and a portion of the annular frame to which the flow conditioner is connected can be formed of a shape-memory alloy.

The shape-memory alloy can be nitinol.

The flow conditioner can be electromechanically actuated.

An attachment angle of the flow conditioner as measured with respect to a longitudinal axis of a respective one of the plurality of struts to which the flow conditioner is connected can be controllable over a range of possible angles.

The flow conditioner can include one or more plates that span across a portion of the annular frame such that the one or more plates intersect a flow axis through the annular frame, the one or more plates each including a plurality of flow passages.

The one or more plates can include a first plate having a first plurality of flow passages and a second plate having a second plurality of flow passages, and the first plate can be connected at an inflow end of the prosthetic valve device to modify the hemodynamic characteristic of the flow of blood through the annular frame and the second plate can be connected to an outflow end of the prosthetic valve device to modify the hemodynamic characteristic of the flow of blood out of the annular frame.

The flow conditioner can be connected to the annular frame at an inflow end of the prosthetic valve device to modify the hemodynamic characteristic of the flow of blood through the annular frame.

The flow conditioner can be connected to the annular frame at an outflow end of the prosthetic valve device to modify the hemodynamic characteristic of the flow of blood out of the annular frame.

The flow conditioner can include a first flow conditioner feature connected to the annular frame at an inflow end of the prosthetic valve device and a second flow conditioner feature connected to the annular frame at an outflow end of the prosthetic valve device, and the first flow conditioner feature can be positioned to modify the hemodynamic characteristic of the flow of blood through the annular frame and the second flow conditioner feature can be positioned to modify the hemodynamic characteristic of the flow of blood out of the annular frame.

The flow conditioner can have a physical dimension that causes the flow conditioner to avoid interaction with the valvular body and/or an adjacent tissue wall.

The flow conditioner can include at least one of a fin and a plate, the plate including a plurality of flow passages.

The prosthetic valve device can be sterilized.

The prosthetic valve device can be configured to be implanted at a valve site.

The prosthetic valve device can be configured to be implanted at a non-valve site.

A prosthetic valve system includes a prestent device having a frame with a bi-directionally flared profile that is formed of a first plurality of struts, a prosthetic valve device configured to sit within the prestent device, a first flow conditioner, and a second flow conditioner. The prosthetic valve device includes an annular frame formed of a second plurality of struts and a valvular body mounted within the annular frame. The valvular body includes a plurality of leaflets that regulate a flow of blood through the annular frame. The first flow conditioner is connected to the first plurality of struts of the prestent device. The first flow conditioner is positioned to modify a first hemodynamic characteristic of the flow of blood through or out of the prestent device. The second flow conditioner is connected to the second plurality of struts of the prosthetic valve device. The second flow conditioner is positioned to modify a second hemodynamic characteristic of the flow of blood through or out of the prosthetic valve device.

The prosthetic valve system of the preceding paragraph can optionally include, additionally and/or alternatively, any one or more of the following features, configurations, and/or additional components:

The first flow conditioner and the second flow conditioner can include one or more fins.

Individual fins of the one or more fins can be connected circumferentially about a respective interior of the frame of the prestent device and/or the annular frame of the prosthetic valve device.

The individual fins of the one or more fins can be angled radially inward from a respective circumference of the frame of the prestent device and/or the annular frame of the prosthetic valve device.

Individual fins of the one or more fins can be connected adjacent to a respective inflow or outflow end of the frame of the prestent device and/or the annular frame of the prosthetic valve device.

The one or more fins can include a first fin connected to the frame at an inflow end of the prestent device to modify the hemodynamic characteristic of the flow of blood through the prestent device, a second fin connected to the frame at an outflow end of the prestent device to modify the hemodynamic characteristic of the flow of blood out of the prestent device, a third fin connected to the annular frame at an inflow end of the prosthetic valve device to modify the hemodynamic characteristic of the flow of blood through the prosthetic valve device, and a fourth fin connected to the annular frame at an outflow end of the prosthetic valve device to modify the hemodynamic characteristic of the flow of blood out of the prosthetic valve device.

Each fin of the one or more fins of the first flow conditioner can be deflectable by the flow of blood through or out of the prestent device, and each fin of the one or more fins of the second flow conditioner can be deflectable by the flow of blood through or out of the prosthetic valve device.

An individual fin of the one or more fins can be connected to the frame of the prestent device or the annular frame of the prosthetic valve device by a spring.

The one or more fins can be airfoils, and the one or more fins can include flow microfeatures proximal to a leading edge of respective ones of the one or more fins.

The flow microfeatures can include at least one of a vortex generator, a leading edge notch, a leading edge dogtooth, a boundary layer fence, and a vortilon.

The first flow conditioner and the frame of the prestent device can form a first monolithic structure, and the second flow conditioner and the annular frame of the prosthetic valve device can form a second monolithic structure.

The first flow conditioner and a portion of the frame of the prestent device to which the first flow conditioner is connected and the second flow conditioner and a portion of the annular frame of the prosthetic valve device to which the second flow conditioner is connected can all be formed of a shape-memory alloy.

The shape-memory alloy can be nitinol.

The first flow conditioner and the second flow conditioner can be electromechanically actuated.

The first flow conditioner can include one or more plates that span across a portion of the frame of the prestent device such that the one or more plates of the first flow conditioner intersect a flow axis through the frame, the second flow conditioner can include one or more plates that span across a portion of the annular frame of the prosthetic valve device such that the one or more plates of the second flow conditioner intersect a flow axis through the annular frame, and each of the one or more plates of the first and second flow conditioners can include a respective plurality of flow passages.

The one or more plates of the first and second flow conditioners can include a first plate connected at an inflow end of the prestent device to modify the hemodynamic characteristic of the flow of blood through the prestent device, a second plate connected at an outflow end of the prestent device to modify the hemodynamic characteristic of the flow of blood out of the prestent device, a third plate connected at an inflow end of the prosthetic valve device to modify the hemodynamic characteristic of the flow of blood through the prosthetic valve device, and a fourth plate connected at an outflow end of the prosthetic valve device to modify the hemodynamic characteristic of the flow of blood out of the prosthetic valve device.

The first flow conditioner can be connected to the frame at an inflow end of the prestent device to modify the hemodynamic characteristic of the flow of blood through the prestent device, or the first flow conditioner can be connected to the frame at an outflow end of the prestent device to modify the hemodynamic characteristic of the flow of blood out of the prestent device.

The second flow conditioner can be connected to the annular frame at an inflow end of the prosthetic valve device to modify the hemodynamic characteristic of the flow of blood through the prosthetic valve device, or the second flow conditioner can be connected to the annular frame at an outflow end of the prosthetic valve device to modify the hemodynamic characteristic of the flow of blood out of the prosthetic valve device.

The first flow conditioner can include one or more fins and the second flow conditioner can include one or more plates that span across a portion of the annular frame of the prosthetic valve device such that the one or more plates of the second flow conditioner intersect a flow axis through the annular frame, or the first flow conditioner can include one or more plates that span across a portion of the frame of the prestent device such that the one or more plates of the first flow conditioner intersect a flow axis through the frame and the second flow conditioner can include one or more fins.

The first flow conditioner and the second flow conditioner can have respective physical dimensions that cause the first and second flow conditioners to avoid interaction with an adjacent tissue wall.

Each of the first flow conditioner and the second flow conditioner can include at least one of a fin and a plate, the plate including a plurality of flow passages.

The prestent device and the prosthetic valve device can be sterilized.

The prosthetic valve system can be configured to be implanted at a valve site.

The prosthetic valve system can be configured to be implanted at a non-valve site.

A cardiovascular implant device includes a body and a flow conditioner connected to the body. The body is configured to attach to one or more leaflets of a natural heart valve. The body includes a central spacer and clasps extending radially outward from the central spacer. Each of the clasps includes a first arm and a second arm for gripping the one or more leaflets. The flow conditioner is positioned to modify a hemodynamic characteristic of a flow of blood around the cardiovascular implant device.

The cardiovascular implant device of the preceding paragraph can optionally include, additionally and/or alternatively, any one or more of the following features, configurations, and/or additional components:

The flow conditioner can include one or more fins.

Each fin of the one or more fins can be deflectable by the flow of blood around the cardiovascular implant device.

Each fin of the one or more fins can be connected to the body by a spring.

The one or more fins can be airfoils, and the one or more fins can include flow microfeatures proximal to a leading edge of respective ones of the one or more fins.

The flow microfeatures include at least one of a vortex generator, a leading edge notch, a leading edge dogtooth, a boundary layer fence, and a vortilon.

The flow conditioner and the body can form a monolithic structure.

The flow conditioner and a portion of the body to which the flow conditioner is connected can be formed of a shape-memory alloy.

The shape-memory alloy can be nitinol.

The flow conditioner can be electromechanically actuated.

The flow conditioner can be connected to the body at an inflow end of the cardiovascular implant device.

The flow conditioner can be connected to the body at an outflow end of the cardiovascular implant device.

The flow conditioner can include a first flow conditioner feature connected to the body at an inflow end of the cardiovascular implant device and a second flow conditioner feature connected to the body at an outflow end of the cardiovascular implant device.

The flow conditioner can be connected to the body at the central spacer.

The flow conditioner can be connected to the body at the clasps.

The flow conditioner can have a physical dimension that causes the flow conditioner to avoid interaction with an adjacent tissue wall.

The cardiovascular implant device can be sterilized.

The cardiovascular implant device can be an edge-to-edge valve repair device.

While the invention has been described with reference to an exemplary example(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular example(s) disclosed, but that the invention will include all examples falling within the scope of the appended claims.

1. A cardiovascular implant device for modifying a blood flow into a right side of a human heart, the cardiovascular implant device comprising:
an expandable annular frame formed of a plurality of struts and configured to be placed in a superior vena cava, wherein the expandable annular frame includes a central flow path extending through the expandable annular frame;
a flow conditioner projecting radially inward into the central flow path of the expandable annular frame, wherein the flow conditioner is positioned for modifying a hemodynamic characteristic of the blood flow through or out of the expandable annular frame; and
an electromechanical control system configured to adjust a position of the flow conditioner to modify the blood flow through the central flow path.

2. The cardiovascular implant device of claim 1, wherein modifying the blood flow through or out of the expandable annular frame comprises modifying the blood flow into the right side of the heart to enhance cardiovascular hemodynamics.

3. The cardiovascular implant device of claim 1, and further comprising:
a cover for covering one or more portions of the expandable annular frame, wherein the cover forms a seal to limit or prevent blood flow through portions of the expandable annular frame that are covered by the cover.

4. The cardiovascular implant device of claim 1, wherein an inner diameter of the expandable annular frame varies along a length of the expandable annular frame.

5. The cardiovascular implant device of claim 1, wherein the flow conditioner is an elongated projection from the expandable annular frame.

6. The cardiovascular implant device of claim 1, wherein the flow conditioner includes a plurality of flow conditioners projecting radially inward into the central flow path of the expandable annular frame.

7. The cardiovascular implant device of claim 1, wherein the flow conditioner includes a plurality of flow conditioners arranged around a circumference of the expandable annular frame.

8. The cardiovascular implant device of claim 1, wherein physical dimensions of the flow conditioner are configured to modify the hemodynamic characteristic of the blood flow through or out of the expandable annular frame.

9. The cardiovascular implant device of claim 8, wherein the hemodynamic characteristic is selected from the group consisting of a helicity, a vorticity, a velocity, a turbulence, a flow direction, and any combination therefore.

10. The cardiovascular implant device of claim 1, wherein the flow conditioner is configured to add flow resistance to the blood flow through or out of the expandable annular frame.

11. The cardiovascular implant device of claim 1, wherein the cardiovascular implant device is sterilized.

12. The cardiovascular implant device of claim 1, wherein the flow conditioner includes one or more fins.

13. The cardiovascular implant device of claim 12, wherein the one or more fins are connected circumferentially about an interior of the expandable annular frame.

14. A cardiovascular implant device for modifying a blood flow into a right side of a human heart, the cardiovascular implant device comprising:
an expandable annular frame formed of a plurality of struts and configured to be placed in a superior vena cava, wherein the expandable annular frame includes a central flow path extending through the expandable annular frame; and
a flow conditioner projecting radially inward into the central flow path of the expandable annular frame, wherein the flow conditioner is positioned for modifying a hemodynamic characteristic of the blood flow through or out of the expandable annular frame;
wherein the flow conditioner and the expandable annular frame form a monolithic structure.

15. The cardiovascular implant device of claim 14, and further comprising:
an electromechanical control system electrically coupled to the monolithic structure to modify the blood flow through the central flow path.

16. The cardiovascular implant device of claim 14, wherein the flow conditioner and a portion of the expandable annular frame to which the flow conditioner is connected are formed of a shape-memory alloy.

17. The cardiovascular implant device of claim 16, wherein the shape-memory alloy is nitinol.

18. A cardiovascular implant device for modifying a blood flow into a right side of a human heart, the cardiovascular implant device comprising:
an expandable annular frame formed of a plurality of struts and configured to be placed in a superior vena cava, wherein the expandable annular frame includes a central flow path extending through the expandable annular frame;
an actively controlled flow conditioner positioned for modifying a hemodynamic characteristic of the blood flow through or out of the expandable annular frame; and
an electromechanical control system configured to provide the active control to the actively controlled flow conditioner to modify the blood flow through the central flow path.

19. The cardiovascular implant device of claim 18, and further comprising:
an electrical connector extending through a vasculature and connecting the control system to the actively controlled flow conditioner.

20. The cardiovascular implant device of claim 18, wherein a bias member extends between the actively controlled flow conditioner and one or more struts of the plurality of struts.
